(19) United States
(12) Patent Application Publication
(10) Pub. No.: US 2004/0209259 A1
(43) Pub. Date: Oct. 21, 2004

(54) Methods of determining genotypes in duplicated genes and genomic regions

(75) Inventors: Jennifer Dianne Keen-Kim, Irvine, CA (US); Raymond George Fenwick, Laguna Niguel, CA (US)

Correspondence Address: FOLEY & LARDNER, P.O. BOX 80278, SAN DIEGO, CA, 92138-0278, US

(73) Assignee: Quest Diagnostics Investments Incorporated

(21) Appl. No.: 10/417,804

(22) Filed: Apr. 16, 2003

(51) Int. Cl. C12Q 1/68; C12P 19/34

(52) U.S. Cl. 435/6; 435/91.2

(57) ABSTRACT

The present invention provides methods of determining the genotype at a duplicated region of a genome. The methods involve (a) amplifying the DNA, preferably using four amplification primers The exemplary methods produce a first amplicon corresponding to a first distinct region or gene by utilizing a first primer and a second primer which are selected to produce the first amplicon in the presence of the first distinct region or gene, but not in absence of the first distinct region or gene. A second amplicon corresponding to a second distinct region or gene is also produced by utilizing a third primer and a fourth primer which are selected to produce the second amplicon in the presence of the second distinct region or gene, but not in the absence of the second distinct region or gene. A third and/or fourth amplicon corresponding to a hybrid gene or genes is/are also produced if a hybrid gene is present.

### FIELD OF THE INVENTION

The present invention involves the determination of genotypes in duplicated genes and duplicated genomic regions.

### BACKGROUND OF THE INVENTION

The following discussion of the background of the invention is merely provided to aid the reader in understanding the invention and is not admitted to describe or constitute prior art to the present invention.

Congenital Adrenal Hyperplasia (CAH) is characterized by a defect in adrenal steroid biosynthesis causing reduced corticoid production and increased androgen production. CAH manifests in a variety of phenotypic severities, which are broadly classified as Classic and Non-classic disease. Classical disease is more severe, and includes phenotypes such as ambiguous genitalia in females in utero (classic virilization), adrenal crisis, and salt wasting. Non-classic disease includes less severe phenotypes including virilization in childhood, and in women, hirsutism, inconsistent menstruation, and infertility. See, e.g., The Merck Manual of Diagnosis and Therapy, 17^{th} ed., Beers and Berkow, Eds. (1999), pages 2381-82.

Approximately 90% of CAH is caused by 21-hydroxylase deficiency attributable to mutations in the gene coding for 21-hydroxylase, also referred to as CYP21A2. The 21-hydroxylase gene lies on chromosome 6p21.3 among genes that code for proteins determining human leukocyte antigen (HLA) types. The 21-hydroxylase gene locus also has a pseudogene called CYP21A, which is about 30 kilobases (kb) away from CYP21A2. CYP21A is approximately 98% homologous in structure to CYP21A2, but is rendered inactive due to minor differences in the gene as compared to the active CYP21A gene. The proximity and homology of CYP21A2 with CYP21A is thought to predispose the CYP21A2 to crossovers in meiosis between CYP21A2 and CYP21A, which can result in the loss of gene function.

A number of point mutations and small deletions have been identified that deactivate this second copy. These differences include (in order from exons 1-10): P30L, IVS2-13 A/C>G, Δ8 bp, I172N, exon 6 cluster mutation (I1235N, V236E, and M238K), V281L, F306+t, Q318X, and R356W. Transfer of any of these mutations from the pseudogene CYP21A to the functional CYP21A2 can occur through gene conversion or non-homologous recombination, thus inactivating the functional CYP21A2. Another source of mutation at the CYP21 locus is genomic rearrangement resulting in duplication or deletion of entire segments of the gene region. Large 30 kb deletions that fuse CYP21A with CYP21A2 account for approximately 25% of 21-hydroxylase deficiency. These types of mutation at the CYP21 gene locus account for approximately 90% of 21-hydroxylase deficiency. The remaining 10% of 21-hydroxylase deficiency is caused by rare sporadic mutation. The fact that this locus involves an evolutionary gene duplication produces technical difficulties for diagnosing mutation in the functional CYP21A2. Wedell & Luthman, Hum Genet (1993) 91:236-240; Speiser et al., Molecular Genetics and Metabolism, 71: 527-534 (2000); For a review see White & Speiser, Endocrine Reviews 21(3): 245-291 (2000).

The core problem in CAH is the inability of the adrenal glands to make enough cortisol in the non-salt wasting form, or enough cortisol and salt-retaining hormone in the salt-wasting form. Instead of making cortisol, the hormonal raw materials which usually make cortisol are shifted away to make other hormones, specifically male sex hormones (androgens). As a result more androgens are produced than necessary. Before birth, the excess androgens stimulate the growth of the genitalia. When the child is male this does not cause grave problems. However excess androgens in a female with this disorder causes the child's genitalia to have the appearance of a male although the internal genitalia are normal female. Therefore, methods of identifying the genotype of an individual at the CYP21A2 gene would result in early diagnosis and treatment of this disease.

CAH is an example of a disease associated with a duplicated gene; that is, a gene that is represented in multiple copies within a genome. In particular, CAH sequences in the human genome comprise both a “functional” gene that encodes an enzymatically active 21-hydroxylase protein, and a “pseudogene” that contains the inactive gene. Numerous genes are duplicated in the genome of both eukaryotes and prokaryotes. In humans, a number of diseases are associated with recombination between duplicated gene sequences, including Gaucher disease, familial juvenile nephronophthisis, fascioscapulohumeral muscular dystrophy, spinal muscular atrophy, polycystic kidney disease, chronic granulomatous disease, Hunter syndrome, Charcot-Marie Tooth disease (CMT1A)/hereditary neuropathy with liability to pressure palsies, neurofibromatosis, ichthyosis, red-green color blindness, hemophilia A, incontinentia pigmenti, Emery Dreifuss muscular dystrophy, Shwachman-Diamond Syndrome, Williams-Beuren syndrome, Angelman syndrome/15q11.2-q13 Prader-Willi syndrome, DiGeorge/velocardiofacial syndrome, debrisoquine resistance, Chr 22-BL, Chr 22HsPOX2/DGR6 schizophrenia, Smith-Magenis syndrome, Dup 17p11.2 syndrome, cat eye syndrome, Chr 22-BE1, Chr 22-BE2, and Chr 22-BK.

### SUMMARY OF THE INVENTION

The present invention provides methods of determining the genotype of a duplicated region of a genome. While, for the sake of convenience, the duplicated regions are referred to hereinafter as “duplicated genes” in which the distinct gene sequences of the duplication are predisposed to recombination, the skilled artisan will understand that the methods described hereinafter may be applied to duplicated genomes generally to determine the presence or absence of various recombinations or exchanges between the duplicated sequences. The recombination or gene conversion between duplicated regions, and preferably distinct gene sequences, can result in the formation of hybrid sequences (e.g., hybrid genes), with the consequent formation of a new genotype at the region of the chromosome where the recombination or exchange occurred. The present invention provides methods of determining whether a recombination or exchange has occurred, as well as for determining the precise genotype at these regions. Determination of the genotype frequently allows medical practitioners to immediately and correctly diagnose the presence of a particular genetic disease in the individual.

Therefore, in a first aspect the present invention provides methods of determining the genotype of a gene in a sample of DNA. The method involves first amplifying the DNA using four amplification primers to produce a first amplicon corresponding to a first distinct gene by utilizing a first primer and a second primer which are selected to produce the first amplicon in the presence of the first distinct gene, but not in absence of the first distinct gene. A second amplicon corresponding to a second distinct gene is also produced by utilizing a third primer and a fourth primer which are selected to produce the second amplicon in the presence of the second distinct gene, but not in the absence of the second distinct gene. A third amplicon corresponding to a hybrid gene is also produced (if the hybrid gene is present). The hybrid gene contains a portion of the first distinct gene and a portion of the second distinct gene, and the third amplicon is produced by utilizing one of the first primer and second primer and one of the third primer and fourth primer. The third amplicon is produced in the presence of the hybrid gene, but not in absence of the hybrid gene. In another step a determination is made as to which of the first, second, and third amplicons are produced, thereby determining the genotype at the duplicated region of the chromosome in the sample.

In one embodiment the duplicated gene comprises both a functional gene and a pseudogene. In various embodiments the third amplicon corresponds to a fusion gene of the first distinct gene and the second distinct gene, or a rearranged gene of the first distinct gene and the second distinct gene. In these embodiments, a fourth amplicon may also be produced, where the third amplicon corresponds to one possible hybrid gene (e.g., the fusion gene) and the fourth amplicon corresponds to another possible hybrid gene (e.g., the rearranged gene). In a preferred embodiment the first distinct gene and the second distinct gene are an active and inactive form of the duplicated gene. In a particularly preferred embodiment the method includes the steps of contacting one or more of the resulting amplicon(s) with one or more extension primers under conditions where the extension primers are extended by the addition of a distinctively labeled ddNTP from a set of ddNTPs, determining which distinctively labeled ddNTP is present in each of the extended extension primer(s), and correlating the presence of distinctively labeled ddNTPs with a specific genotype at the duplicated region of the chromosome. Each of the extension primers can have a different molecular weight due to the presence of a unique number of nucleotide residues in each extension primer. In a preferred embodiment the ddNTPs comprise a fluorescent label. The ddNTPs preferably include ddATP, ddCTP, ddGTP, and ddTTP, each of which can be labeled with a distinct fluorescent label. In various embodiments the method also includes separating the amplicon(s) produced by agarose gel electrophoresis. In another embodiment the method includes separating the extended extension primer(s) by capillary electrophoresis.

In a preferred embodiment the presence of the third amplicon is associated with a genetic disease, such as any of the following: Gaucher disease, familial juvenile nephronophthisis, fascioscapulohumeral muscular dystrophy, spinal muscular atrophy, polycystic kidney disease, chronic granulomatous disease, Hunter syndrome, Charcot-Marie Tooth disease (CMT1A)/hereditary neuropathy with liability to pressure palsies, neurofibromatosis, ichthyosis, red-green color blindness, hemophilia A, incontinentia pigmenti, Emery Dreifuss muscular dystrophy, Shwachman-Diamond Syndrome, Williams-Beuren syndrome, Angelman syndrome/15q11.2-q13 Prader-Willi syndrome, DiGeorge/velocardiofacial syndrome, debrisoquine resistance, Chr 22-BL, Chr 22HsPOX2/DGR6 schizophrenia, Smith-Magenis syndrome, Dup 1 7p11.2 syndrome, cat eye syndrome, Chr 22-BE1, Chr 22-BE2, and Chr 22-BK. This list is not meant to be limiting, and other types of genetic diseases caused by genetic rearrangements may be identified by the methods described herein.

By a “distinct gene” is meant a sequence of nucleotides in a genomic sequence that encodes a functional messenger RNA molecule, or a pseudogene corresponding to such a sequence. Thus two variations of a gene may be referred to as a “first distinct gene” and a “second distinct gene.” In some cases the second distinctive gene is an inactive form of the gene, such as a pseudogene, while in other cases it may retain some or all activity, or have a slightly different activity from the original copy. In still other cases the second distinct gene may not be a complete copy, but rather a portion of the original copy with the remainder lost to a genetic deletion or other type of genetic rearrangement. As noted above, the present invention is not limited to duplicated genes in which the distinct gene sequences of the duplication are predisposed to recombination, the skilled artisan will understand that the methods described hereinafter may be applied to duplicated genomes generally to determine the presence or absence of various recombinations or exchanges between the duplicated sequences. Thus, references herein that refer to a “first distinct gene” and a “second distinct gene” may be considered equally applicable to a first copy of a duplicated genomic sequence and a second copy of a duplicated genomic sequence.

A gene (or genomic region) is said to be duplicated if a second distinct gene (or second copy) is at least 85% identical, more preferably at least 90% identical, and most preferably or at least 95% identical to a first distinct gene (or first copy) over one or more stretches of at least 100 contiguous nucleotides, preferably at least 200 contiguous nucleotides, more preferably at least 5000 contiguous nucleotides, and most preferably at least 1000 contiguous nucleotides. Protein identity is determined by aligning two sequences using BLAST (Altschul, et al., “Gapped BLAST and PSI-BLAST: a new generation of protein database search programs.” Nucleic Acids Res. 25:3389-3402(1997)) with default parameters. Most preferably, the second distinctive gene is at least 85% identical, more preferably at least 90% identical, and most preferably or at least 95% identical to the first distinctive gene over their common length.

A duplicated gene is said to be “predisposed to recombination” if the first distinct gene and the second distinct gene exist in recombined form in the general population at a rate of at least 0.00001%; that is 1 in 100,000 individuals carry a hybrid gene. More preferably, a duplicated gene that is predisposed to recombination exist in recombined form in the general population at a rate of at least 0.0001%, and even more preferably at least 0.001%.

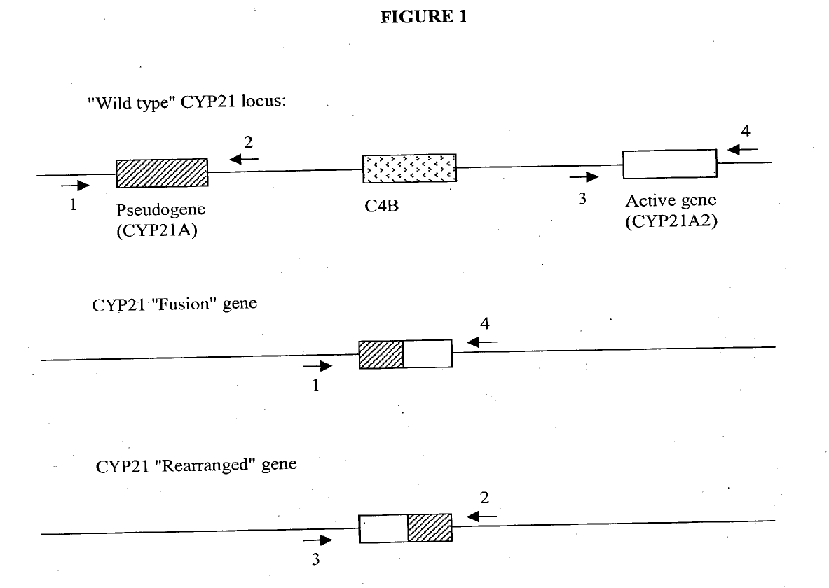

A “hybrid” gene refers to a genetically rearranged gene resulting in the whole or partial fusion of two distinct genes. The term “fusion gene” as used herein refers to a hybrid gene created by joining portions of two different genes, e.g., the CYP21A2 and CYP21A genes, without altering the relative 3′-to-5′ order of the fused sequences. A fusion gene can result from the deletion of a nucleic acid sequence in between two genes, thus causing the two genes to become “fused” without altering the 3′-to-5′ order of the sequences remaining in the fused gene. In the present invention a nucleic acid sequence in between the CYP21A2 and CYP21A genes is sometimes deleted, causing the formation of a fusion gene, which contains parts of the CYP21A2 and CYP21A genes. FIG. 1 depicts an example of a fusion gene. Sequences upstream from the CYP21A gene can remain upstream of the fusion gene, and sequences downstream from the CYP21A2 gene can remain downstream of the fusion gene, providing binding sequences for the primers of the present invention.

Similarly, the term “rearranged gene” as used herein refers to a hybrid gene created by joining portions of two different genes, e.g., the CYP21A2 and CYP21A genes, in which the relative 3′-to-5′ order of the fused sequences is altered. In the present invention, the “first half” of the CYP21A2 gene can become fused to the “second half” of the CYP21A gene, causing the formation of a rearranged gene, which contains parts of the CYP21A2 and CYP21A genes. FIG. 1 depicts an example of a rearranged gene. Sequences upstream from the CYP21As gene can remain upstream of the rearranged gene, and sequences downstream from the CYP21A gene can remain downstream of the rearranged gene, providing binding sequences for the primers of the present invention.

The skilled artisan will understand that duplicated regions of the genome are very dynamic. For example, hybrid genes can also arise by gene conversion (e.g., genetic change without exchange of flanking markers). Thus, hybrid genes may arise with or without the concomitant loss of a “parent” duplicated gene sequence. See, e.g., Chung et al., Am. J. Hum. Genet. 71(4):823-37 (2002).

The sample analyzed in the present methods is a sample of DNA, preferably a sample of genomic DNA. By “genomic DNA” is meant a sample of DNA from an organism that contains coding and noncoding (intron and other) sequences of DNA of the organism. For example, a sample of genomic DNA may be obtained from a blood sample by isolating the DNA from nucleated cells in the sample.

Any sample comprising genomic DNA may be used as the source of material to be genotyped. The term “biological sample” as used herein refers to a sample obtained from a biological source, e.g., an organism, cell culture, tissue sample, etc. A biological sample can, by way of non-limiting example, consist of or comprise blood, sera, urine, feces, epidermal sample, skin sample, cheek swab, sperm, amniotic fluid, cultured cells, bone marrow sample and/or chorionic villi. Biological samples may be obtained from plants, animals, fungi, etc.

The term “subject” as used herein refers to any eukaryotic organism. Preferred subjects are fungi, plants, invertebrates, insects, arachnids, fish, amphibians, reptiles, birds, marsupials and mammals. A mammal can be a cat, dog, cow, pig, horse, ox, elephant, simian. Most preferred subjects are humans. A subject can be a patient, which refers to a human presenting to a medical provider for diagnosis or treatment of a disease. Methods to study the role of cytochrome P450 enzymes in metabolism in animals are known (see, e.g., Chauret et al., In Vitro Comparison of P450-Mediated Metabolic Activities in Human, Dog, Cat, and Horse, Drug Metabolism and Disposition 25:1130-1136, 1997). The term “animals” includes metamorphic and prenatal forms of animals.

In the disclosure, a “plurality of samples” refers to at least two. Preferably, a plurality refers to a relatively large number of samples. A plurality of samples is from about 5 to about 500 samples, preferably about 25 to about 200 samples, most preferably from about 50 to about 200 samples. Samples that are processed in a single batch run of the method of the invention are usually prepared in plates having 24, 48, 96, 144, or 192 wells. The term “samples” includes samples per se as well as controls, standards, etc. that are included in a batch run.

The term “amplifying” as used herein refers to an increase in the copy number of nucleic acid sequences. Nucleic acid sequences can be amplified as necessary for further use using amplification methods, such as PCR, isothermal methods, rolling circle methods, etc., well known to the skilled artisan. See, e.g., Saiki, “Amplification of Genomic DNA” in PCR Protocols, Innis et al., Eds., Academic Press, San Diego, Calif. 1990, pp 13-20; Wharam et al., Nucleic Acids Res. 2001 Jun. 1;29(11):E54-E54; Hafner et al., Biotechniques April 2001;30 (4):852-6, 858, 860 passim; Zhong et al., Biotechniques April 2001;30(4):852-6, 858, 860 passim. Amplification is preferably performed by the polymerase chain reaction (PCR). The “polymerase chain reaction” (or PCR amplification) is a method for copying and amplifying specific sequences of nucleotides in DNA using a heat-stable polymerase and two nucleic acid primers, one complementary to the (+)-strand at one end of the sequence to be amplified and the other complementary to the (−)-strand at the other end. Because the newly synthesized DNA strands can subsequently serve as additional templates for the same primer sequences, successive rounds of primer annealing, strand elongation, and dissociation produce rapid and highly specific amplification of the desired sequence. PCR also can be used to detect the existence of the defined sequence in a DNA sample.

The term “amplicon” as used herein refers to a product of an amplification reaction that is a copy of a particular DNA sequence.

In some embodiments a control amplicon is also generated as a control for the PCR reaction. Typically, a control amplicon may be generated from a sequence known to be present in the DNA sample. Such sequences are often “housekeeping” genes, but may be any genomic sequence that is unrelated to the genomic sequence of interest. The control amplicon is generated in the absence of any of the test amplicons to identify false negatives; that is, when an amplicon is absent, it may be due to either the absence of the target sequence, or may be due to a failure of the entire amplification reaction.

Nucleic acid “primers” are oligonucleotides that bind to and direct polymerization activity at a particular region of a nucleic acid molecule. In PCR, primers define the area of the genome to be amplified by binding to it and directing the activity of DNA polymerase to that sequence.

The term “distinctively labeled” as used herein refers to each member of a set that is labeled with a distinct label so that each member can be distinguished from the other members. For example, in a set of distinctively labeled nucleotides (e.g., dideoxy NTPs, or ddNPTs), each type of “N” (nucleotide) is labeled with a label that can be distinguished from the other types of labels. Thus, for example, if four labels designated 1, 2, 3, and 4 are used to label the four types of ddNTPs, each ddATP molecule is labeled with label “*1”, each ddTTP molecule is labeled with label “*2”, each ddCTP molecule is labeled with label “*3”, and each ddGTP molecule is labeled with label “*4”. In some embodiments of the invention, the distinctive label is a fluorescent label. In preferred embodiments the signal is provided by each ddNTP having a different color.

In another aspect, the present invention is described in terms of methods for determining a 21-hydroxylase (CYP21) genotype in a sample of DNA. The methods involve amplifying genomic DNA to produce one or more amplicons from the CYP21 locus. By careful selection of amplification primers, four primers may be used to generate the following amplicons (i) a first amplicon corresponding to an intact CYP21A pseudogene, if present, utilizing a first primer and a second primer that produce the first amplicon in the presence of the intact pseudogene but not in the absence of the intact pseudogene; (ii) a second amplicon corresponding to an intact CYP21A2 gene, if present, by utilizing a third primer and a fourth primer that produces the second amplicon in the presence of the intact CYP21A2 gene but not in absence of the intact CYP21A2 gene. Two of the primers also amplify a CYP21A/CYP21A2 fusion gene if present to produce a third amplicon and two of the primers amplify a CYP21A2/CYP21A rearranged gene if present to produce a fourth amplicon. A schematic description of exemplary primers for use in this method is provided in FIG. 1.

The skilled artisan will understand that, for each allele of the CYP21 gene locus, the presence of the fusion gene can occur in the presence or absence of the intact linked CYP21A pseudogene, intact linked CYP21A2 gene, and the linked CYP21A2/CYP21A rearranged gene; likewise the presence of the rearranged gene can occur in the presence or absence of the intact CYP21A linked pseudogene, intact linked CYP21A2 gene, and the linked CYP21A/CYP21A2 fusion gene; and the intact pseudogene or active gene can occur in the presence or absence of the linked CYP21A/CYP21A2 fusion gene and the linked CYP21A2/CYP21A rearranged gene. The four amplification primers described herein, and their equivalents, can be used to identify the presence or absence of the fusion gene, the rearranged gene, the intact pseudogene, and the intact active gene.

The resulting amplicons are preferably then used for the subsequent analysis of one or more predetermined single nucleotide polymorphisms (“SNPs”). In preferred embodiments, this analysis comprises contacting one or more of the amplicon(s) produced with one or more extension primers selected to indicate the presence of a predetermined single nucleotide polymorphism under conditions where the extension primers are extended by the addition of a distinctively labeled ddNTP from a set of ddNTPs. The identity of the distinctively labeled ddNTP added to each of the extended extension primer(s) can be used to determine the presence or absence of one or more CYP21A2 SNPs. Alternative methods for analyzing SNPs include flow cytometric methods; sequencing methods; array hybridization; mismatch detection; restriction/digestion, mass spectrometry methods, etc. See, e.g., Taylor et al., Biotechniques 30:661-6, 668-₉ (2001); Landegren et al., Genome Research 8: 769-76 (1998); and U.S. Pat. No. 6,043,031; each of which is hereby incorporated in their entirety.

In various embodiments each of the extension primers used in the present methods is designed to have a different molecular weight, and the extension primers are extended by the addition of one nucleotide through a single base extension reaction. The different molecular weights may be generated by the presence of one or more of nucleotides attached to the extension primers that do not hybridize to the CYP21 gene locus. For example, a stretch of poly-T, poly-A, poly-C, or poly-G may be added to each extension primer in the extension primer set to generate different molecular weights. In this manner, each extension primer may be identified by its size, and hence its migration distance or rate in a separation system (e.g., a capillary electrophoresis system).

In a particularly preferred embodiment the ddNTPs contain a detectable label, most preferably a fluorescent label, and the set of ddNTPs contains ddATP, ddCTP, ddGTP, and ddTTP, each of which is labeled with a distinct detectable label. Preferred detectable labels are described hereinafter.

In preferred embodiments, the amplification primers are selected so that first amplicon is about 4.0 kb in length, corresponding to the CYP21A pseudogene; the second amplicon is about 3.4 kb in length corresponding to the CYP21A2 functional gene; the third amplicon is about 4.0 kb in length corresponding to the CYP21A/A2 fusion gene; and the fourth amplicon is about 3.4 kb in length corresponding to the CYP21A2/A rearrangement gene. In the most preferred embodiment the first and second primers flank the CYP21A pseudogene if present; and the third and fourth primers flank the CYP21A2 active gene if present; the first and fourth primers flank the CYP21A/CYP21A2 fusion gene if present; and the third and second primers flank the CYP21A2/CYP21A rearranged gene if present. The term “about” as used herein means ±15%. These embodiments are exemplary only, and the skilled artisan will understand that numerous primers could be selected to provide a variety of differently sized amplicons.

In particularly preferred embodiments, the primers selected have one or more of the following sequences: the first primer has the sequence <SEQ ID NO 1> TCCCCAATCCTTACTTTTTGTC; the second primer has the sequence <SEQ ID NO 2> CCTCAATCCTCTGCGGCA; the third primer has the sequence <SEQ ID NO 3> GCTTCTTGATGGGTGATCAAT; and the fourth primer has the sequence <SEQ ID NO 4> CCTCAATCCTCTGCAGCG. The skilled artisan will understand that complementary or conservative sequences thereof may also be used.

A “complementary sequence” as used herein refers the opposing strand of a nucleic acid duplex, corresponding to Watson-Crick base pairing rules. Thus, a strand having an A, T, G, and C in particular locations on the strand would have T, A, C, and G on the complementary strand. Conservative sequences are sequences having at least 60% homology to the sequence of interest, in this case a nucleic acid primer. In other embodiments conservative sequences have at least 70% homology or at least 75% homology or at least 80% homology at least 85% homology at least 90% homology or at least 95% homology or at least 98% homology. For purposes of the present invention, the degree of homology between two nucleic acid sequences is determined by using GAP version 8 from the GCG package with standard penalties for DNA: GAP weight 5.00, length weight 0.300, Matrix described in Gribskov and Burgess, Nucl. Acids Res. 14(16); 6745-6763 (1986).

In preferred embodiments, the determination of which amplicons are produced is determined by agarose gel electrophoresis, for example on a 0.7% agarose slab gel. In still other preferred embodiments, the determination of which distinctively labeled ddNTP is present in each of the extended extension primer(s) is determined by capillary electrophoresis. In particularly preferred embodiments, capillary electrophoresis is coupled with fluorescence detection to detects the distinctively labeled ddNTP present.

In various embodiments, the extension primers used bind to one or more sequences characteristic of a genotype selected from: IVS2-13 A/C>G, I172N, V281L, Q318X, R356W, I235N, V236E, M238K, F306+t, Δ8 bp-R, P30L, Δ8 bp-F, and P453S.

As used herein, “primer extension” refers to the enzymatic extension of the three-prime (3′) hydroxy group of an extension primer, which is an oligonucleotide X nucleotides long that is paired to a template nucleic acid (for an example of primer extension as applied to the detection of polymorphisms, see Fahy et al., Multiplex fluorescence-based primer extension method for quantative mutation analysis of mitrochondrial DNA and its diagnostic application for Alzheimer's disease, Nucleic Acid Research 25:3102-3109, 1997). The extension reaction is catalyzed by a DNA polymerase. Primer extension reactions can be “single base extensions” where the primer is extended by a single base. By “DNA Polymerase” is meant a DNA polymerase, or a fragment thereof, that is capable of carrying out primer extension. Thus, a DNA polymerase can be an intact DNA polymerase, a mutant DNA polymerase, an active fragment from a DNA polyerase, such as the Klenow fragment of E. coli DNA polymerase, and a DNA polymerase from any species including, but not limited to, thermophiles.

Extension of the 3′ end of the oligonucleotide generates an oligonucleotide having a length of at least (X+Y) nucleotides, where Y≧ one, having a sequence that is the reverse complement of the template nucleic acid. If one of the nucleotides in the added sequence Y is labeled, then the extended (X+Y) oligonucleotide is labeled.

For example, in the following diagram of a primer extension reaction, four different ddNTPs, each distinctively labeled, are present in the reaction mixture as designated by dd(A*1)TP, dd(T*2)TP, dd(C*3)TP and dd(G*4)TP, where *1, *2, *3 and *4 represent different labels. In the diagram, the polymorphism in the nucleic acid being tested is indicated by an underlined nucleotide, and the extension primer sequence is italicized. Only one ddNTP, ddTTP, can be added to the 3′ end of the extension primer, because thymine (T) is the only base that pairs with adenosine (A). The addition of the dd(T*2)TP to the 3′ of the primer prevents any further primer extension because it is a dideoxy, chain-terminating ddNTP. Thus, the only primer that is 3′ extended is labeled with label *2. Detection of the signal from label *2 indicates that the A polymorphism is present in the sample.

| | |
|---|---|
| wildtype | 5′ CCGGGGTGGTTGGCGAAGGCAGTCCCCTG |
| | TGCTGCC-3′ |
| sample | 5′ CCGGAGTGGTTGGCGAAGGCAGTCCCCTG |
| | TGCTGCC-3′ |
| | \|\|\|\|\|\|\|\|\|\|\|\|\|\|\|\|\|\|\|\|\|\| |
| primer | 3′ CACCAACCGCTTCCGTCAGTGGA-5′ |
| labeled ddNTP | |
| dd(A^{*1})TP | 3′ CACCAACCGCTTCCGTCAGTGGA-5′ |
| dd(T^{*2})TP | 3′ ^{*2}TCACCAACCGCTTCCGTCAGTGGA-5′ |
| dd(C^{*3})TP | 3′ CACCAACCGCTTCCGTCAGTGGA-5′ |
| dd(G^{*4})TP | 3′ CACCAACCGCTTCCGTCAGTGGA-5′ |

In preferred embodiments, each extension primer has a nucleotide sequence that binds in a complementary fashion to a portion of a sequence of nucleic acid that represents the genotype at the CYP21 gene. Preferred extension primers are of a length sufficient to provide specific binding to the sequence of interest. Such primers comprise an exact complement to the sequence of interest for 10 to 40 nucleotides in length, and preferably 20 to 30 nucleotides in length. The extension primer sequence has a 3′ terminus that pairs with a nucleotide base that is, in the sample nucleic acid to which the primer is hybridized, 5′ from the site of one or more bases in the sequence of interest that represent a polymorphism in a gene. In addition to this sequence the primer in the preferred embodiments has a polymer “tail,” or portion that distinguishes the primer based on molecular weight. The polymer is preferably a poly-thymine tail from 2 or 3 nucleotides in length up to any individual number of nucleotides, such as about 50 or 75 nucleotides or 75-100 nucleotides (or 50-75 nucleotides or 75-100 nucleotides or greater than 100 nucleotides) in length or even a greater number of nucleotides. Exemplary extension primers for use in CYP21 genotyping are disclosed, e.g., in Krone et al., Clin. Chem. 48(6) 818-825 (2002).

An amount of nucleic acid sufficient for primer extension can, but need not be, prepared by amplification via polymerase chain reaction (PCR) using PCR primers. As a non-limiting example, when the preselected CAH gene is CYP21A, an appropriate PCR primer includes, but is not limited to, one or more, and preferably all, of those having sequences selected from the group provided in the table below:

| | |
|---|---|
| <SEQ 1D NO 5> | TTTTCCCGGGGCAAGAGGC; |
| <SEQ ID NO 6> | TTTCCAGCTTGTCTGCAGGAGGAG; |
| <SEQ ID NO 7> | TTTTTTTTTTTCTCCGAAGGTGAGGTAACAG; |
| <SEQ ID NO 8> | TTTTTTTTTTTTTTTTTGGACAGCTCCTGGAAGG |
| | GCAC; |
| <SEQ ID NO 9> | TTTTTTTTTTTTTTTTTTTTTTTTTCCCCAGATT |
| | CAGCAGCGACTG; |
| <SEQ ID NO 10> | TTTTTTTTTTTTTTTTTTTTTTTTTTTTTTTTAT |
| | CGCCGAGGTGCTGCGCCTG; |
| <SEQ ID NO 11:> | TTTTTTTTTTTTTTTTTTTTTTTTTTTTTTTTTT |
| | TTTTTTCCATAGAGAAGAGGGAYCACA; |
| <SEQ ID NO 12: | TTTTTTTTTTTTTTTTTTTTTTTT TTTTTTTTT |
| | TTTTTTTTTTTTGCTGCCTCAGCTGCWTCTCC; |
| <SEQ ID NO: 13> | TTTTTTTTTTTTTTTTTTTTTTTTTTTTTTTTTT |
| | TTTTTTTTTTTTTTCCTTGTGCTGCCTCAGCTGC; |
| <SEQ ID NO: 14> | TTTTTTTTTTTTTTTTTTTTTTTTTTTTTTTTTT |
| | TTTTTTTTTTTTTTCACCCTCTCCTGGGCCGTGG |
| | TTTTTTT; |
| <SEQ ID NO: 15> | TTTTTTTTTTTTTTTTTTTTTTTTTTTTTTTTTT |
| | TTTTTTTTTTTTTTTTTTTTTTTTTTTTTTACCC |
| | GGACCTGTCSTTG; |
| <SEQ ID NO 16> | TTTTTTTTTTTTTTTTTTTTTTTTTTTTTTTTTT |
| | TTTTTTTTTTTTTTTTTTT TTTTTTTTTTTTTG |
| | GGCTTTCCAGAGCA; |
| <SEQ ID NO 17> | TTTTTTTTTTTTTTTTTTTTTTTTTTTTTTTTTT |
| | TTTTTTTTTTTTTTTTTTTTTTTTTTTTTTTTTT |
| | TTTTTTTCAGGCCTTCACGCTGCTG. |

For each reaction mixture, the amount of the nucleic acid sufficient for primer extension or for performing PCR amplification is determined by obtaining a sample comprising nucleic acid and determining the concentration of nucleic acid therein. One skilled in the art will be able to prepare such samples to a concentration and purity necessary to practice the invention, and to estimate the amount of a specific sample that should be added to a particular reaction mixture. A failure to detect a signal in the method of the invention signifies that, among other things, an inadequate amount of nucleic acid has been added to a reaction mixture. Those skilled in the art will be able to trouble-shoot failed batch runs and adjust the contents of the reaction mixtures and/or conditions of the run accordingly. Control samples can be included in the batch runs to confirm that appropriate amounts of nucleic acid are present.

One or more of steps of the foregoing methods, or combinations thereof, are preferably performed automatically, typically using robotics, in order to provide for the processing of a large number of samples in a single batch run. Preferred forms of automation will provide for the preparation and separation of a plurality of labeled nucleic acids in small volumes. The term “small volumes” refers to volumes of liquids less than 2 ml, e.g., any volume from about 0.001 picoliters or about 0.001 μl, to any volume of about 2 ml, 500 μl, 200 μl, 100 μl, 10 μl, 1 μl, 0.1, 0.2 μl, μl, 0.01 μl, or 0.001 μl.

After primer extension, the set of distinctively labeled polynucleotides can be separated from each other so that each is mobilized in a manner that relates to each of their specific positions in the respective nucleotide sequence, and the detection of the distinctive signals generated from the distinctively labeled polynucleotides occurs during or after the mobilization step. The distinctively labeled polynucleotides can be separated from each other by electrophoresis. A preferred form of electrophoresis is capillary electrophoresis, or any form of electrophoresis that allows for the separation of a plurality of labeled nucleic acids in small volumes by automated or semi-automated methods and devices.

The CYP21 mutations or variants can be of any type, including, but not limited to, deletions, inversions, insertions, translocations, mutations resulting in aberrant RNA splicing, single nucleotide polymorphisms, and combinations thereof. The CYP21 mutations include, but are not limited to, those reflected in Table 1 below.

In another aspect the present invention provides a substantially purified nucleic acid sample comprising one or more nucleic acids having sequences selected from <SEQ ID NO 1> TCCCCAATCCTTACTTTTTGTC, <SEQ ID NO 2> CCTCAATCCTCTGCGGCA, <SEQ ID NO 3> GCTTCTTGATGGGTGATCAAT, and <SEQ ID NO 4> CCTCAATCCTCTGCAGCG, or a complementary or conservative nucleic acid sequence thereof, in an amount sufficient to perform a polymerase chain reaction amplification of a nucleic acid sample.

In preferred embodiments, one or more of the sequences are present at a concentration of about 100 nM, but the person or ordinary skill will realize that lower or higher concentrations of primers will also serve to accomplish PCR, such as concentrations of 150 nM, or more, 50 nM 25 nM, 10 nM and even less. Furthermore, specific conditions of PCR can be utilized (e.g., longer cycling times) that will enable the usage of lower concentrations. A nucleic acid sample is amplified when the number of copies is increased by at least 1,000 times. More preferably the number of copies is increased by at least 10,000 times or at least 100,000 times or at least 1,000,000 times.

In another aspect the present invention provides a substantially purified nucleic acid sample containing one or more, and preferably each of the nucleic acids selected from the following group:

| | |
|---|---|
| <SEQ ID NO 5> | TTTTCCCGGGGCAAGAGGC; |
| <SEQ ID NO 6> | TTTCCAGCTTGTCTGCAGGAGGAG; |
| <SEQ ID NO 7> | TTTTTTTTTTTCTCCGAAGGTGAGGTAACAG |
| <SEQ ID NO 8> | TTTTTTTTTTTTTTTTTGGACAGCTCCTGGAAGG |
| | GCAC; |
| <SEQ ID NO 9> | TTTTTTTTTTTTTTTTTTTTTTTTTCCCCAGATT |
| | CAGCAGCGACTG; |
| <SEQ ID NO 10> | TTTTTTTTTTTTTTTTTTTTTTTTTTTTTTTTAT |
| | CGCCGAGGTGCTGCGCCTG; |
| <SEQ ID NO 11:> | TTTTTTTTTTTTTTTTTTTTTTTTTTTTTTTTTT |
| | TTTTTTCCATAGAGAAGAGGGAYCACA; |
| <SEQ ID NO 12: | TTTTTTTTTTTTTTTTTTTTTTTT TTTTTTTTT |
| | TTTTTTTTTTTTGCTGCCTCAGCTGCWTCTCC; |
| <SEQ ID NO: 13> | TTTTTTTTTTTTTTTTTTTTTTTTTTTTTTTTTT |
| | TTTTTTTTTTTTTTCCTTGTGCTGCCTCAGCT |
| | GC; |
| <SEQ ID NO: 14> | TTTTTTTTTTTTTTTTTTTTTTTTTTTTTTTTTT |
| | TTTTTTTTTTTTTTCACCCTCTCCTGGGCCGTGG |
| | TTTTTTT; |
| <SEQ ID NO: 15> | TTTTTTTTTTTTTTTTTTTTTTTTTTTTTTTTTT |
| | TTTTTTTTTTTTTTTTTTTTTTTTTTTTTTACCC |
| | GGACCTGTCSTTG; |
| <SEQ ID NO 16> | TTTTTTTTTTTTTTTTTTTTTTTTTTTTTTTTTT |
| | TTTTTTTTTTTTTTTTTTT TTTTTTTTTTTTTG |
| | GGCTTTCCAGAGCA; |
| <SEQ ID NO 17> | TTTTTTTTTTTTTTTTTTTTTTTTTTTTTTTTTT |
| | TTTTTTTTTTTTTTTTTTTTTTTTTTTTTTTTTT |
| | TTTTTTTCAGGCCTTCACGCTGCTG; |

or a complementary or conservative nucleic acid sequences of any of these. While the above primers pertain specifically to CAH, the person of ordinary skill will realize, with reference to the present disclosure, that primers specific for genotypes and mutations specific for other genetic diseases can also be designed using the same principles.

In another aspect the present invention provides a kit containing one or more nucleic acids or sets of nucleic acids as described above in an amount sufficient to perform a polymerase chain reaction amplification of a nucleic acid sample and/or a single base extension reaction on a nucleic acid sample. The kits optionally contain reagents for performing a PCR amplification and/or a single base extension reaction, wherein the nucleic acids and reagents, if present, are provided in a container. The container is preferably a box or wrapping that encloses the items of the kit. In one embodiment the kit contains one or more nucleic acids of <SEQ ID NOS. 1-4>. In another embodiment the kit contains one or more nucleic acids of <SEQ ID NOS. 5-17>. The person of ordinary skill will realize with reference to the present disclosure that kits specific for the detection of other genetic diseases, or for a battery of genetic diseases, can be designed using the same principles.

In various aspects of the present invention, the genotyping methods described herein may be used to identify subjects at risk for diseases related to congenital adrenal hyperplasia. These methods may also be applied to determine carrier status (heterozygosity). The skilled artisan will understand that such methods may be applied to samples from individuals and for prenatal determinations.

In various other aspects, the invention provides a method for selecting a treatment regimen for a particular subject, based upon the identified 21-hydroxylase (CYP21) genotype of the subject. The methods comprise determining a genotype according to the foregoing methods, and selecting a treatment regimen known in the art to ameliorate one or more effects of the genotype identified. A “treatment regimen” is a course of treatment that may include, but is not limited to, drug therapy, changes to lifestyle, changes to diet, surgical intervention, etc. Methods for treatment of 21-hydroxylase deficiency are well known in the art. For example, 21-hydroxylase deficiency is the most frequent cause of ambiguous genitalia in the newborn female. The concordance between genotype and phenotype is sufficiently robust as to be relevant and useful in planning treatment strategies. Hughes, Semin. Reprod. Med. 20(3):229-42 (2002). The dose of glucocorticoid replacement in the early years of life can be tailored according to the predicted degree of 21-hydroxylase enzyme deficiency in the anticipation that this may avoid hitherto excessive steroid replacement during the critical early years of growth and development. The means to prevent genital virilization in affected females is clearly demonstrated by the success of early dexamethasone administration to pregnant mothers at risk. Moreover, individuals with salt losing 21-hydroxylase enzyme deficiency need to replace the lack of aldosterone, typically using Fludrocortisone in a dose of 50-300 micrograms. The correct level of fludrocortisone is determined by measuring blood pressure, potassium and the salt sensitive hormone renin in the blood.

In yet other preferred embodiments, the invention provides a method for selecting one or more subjects for inclusion in a clinical trial, based upon the identified 21-hydroxylase (CYP21) genotype of the subject(s). The methods comprise determining a genotype according to the foregoing methods, and selecting those patients to be included or excluded from the trial based, at least in part, on the genotype identified. In these embodiments, subjects may be excluded or included from the trial, according to their heightened risk of suffering from a disease and/or their predicted responsiveness to a particular treatment regimen. The person of ordinary skill will realize with reference to the present disclosure that treatment regimens specific for treating other genetic diseases can be designed using the same principles.

The summary of the invention described above is non-limiting and other features and advantages of the invention will be apparent from the following detailed description of the invention, and from the claims.

### DETAILED DESCRIPTION OF THE DRAWINGS

FIG. 1 provides a schematic illustration of the CYP21 gene area. Amplification primers are depicted as arrows, indicating the direction in which each primer initiates DNA polymerization.

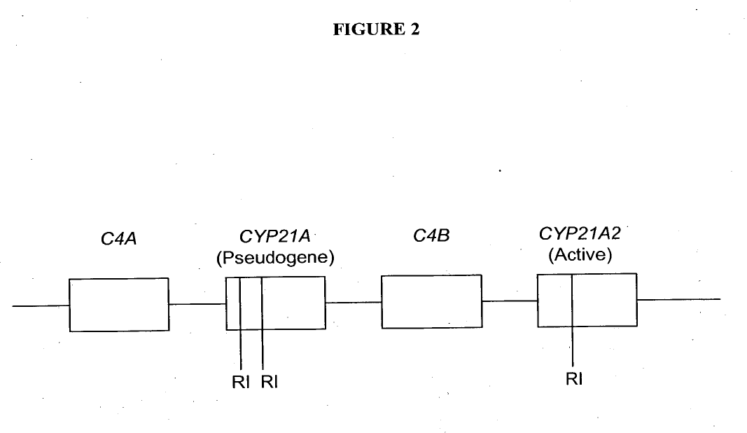

FIG. 2 provides a schematic illustration of the CYP21A and CYP21A2 gene areas. EcoRI restriction enzyme cleavage sites are labeled with “RI”.

### DETAILED DESCRIPTION OF THE INVENTION

Many types of genetic diseases are ultimately due to a duplicated and rearranged gene. For example, each of the following diseases are correlated with duplicated genes. The following provides a non-limiting list of the duplicated genes associated with the disease indicated:

| | |
|---|---|
| CYP21 | Congenital adrenal hyperplasia |
| GBA | Gaucher Disease |
| NPHP1 | Familial juvenile nephronophthisis |
| FRG1 | Fascioscapulohumeral muscular dystrophy |
| SMN | Spinal muscular atrophy |
| PKD1 | Polycystic kidney disease |
| PMP22 | Charcot-Marie Tooth disease |
| | (CMT1A)/Hereditary neuropathy with |
| | liability to pressure palsies |
| NF1 | Neurofibromatosis |
| STS | Ichthyosis |
| RCP/GCP | Red-green color blindness |
| Factor VIII | Hemophilia A |
| NEMO | Incontinentia pigmenti |
| EMD/FLN1 | Emery Dreifuss muscular dystrophy |
| ELM/GTF2I | Williams-Beuren syndrome |
| UBE3A | Angelman syndrome/15q11.2-q13 |
| | Prader-Willi syndrome |
| TBX1 | DiGeorge/Velocardiofacial syndrome |
| CYP2D6 | Debrisoquine resistance |
| BK126B4.1 | Chr 22—BL |
| HsPOX2/DGR6 | Chr 22HsPOX2/DGR6 Schizophrenia |
| SMS del17p11.2 | Smith-Magenis syndrome |
| Dup 17p11.2 | Dup 17p11.2 syndrome |
| Rearrangement 22q11 | Cat Eye Syndrome |
| None | Chr 22—BE1 |
| None | Chr 22—BE2 |
| None | Chr 22—BK |

In addition to the above, many duplicated genes exist but may or may not be associated with a particular genetic disease. Example 2 describes genes that are known to be duplicated and which may find use in the present invention. The invention is described hereinafter using Congenital Adrenal Hyperplasia as an example; however, the skilled artisan will understand that the methods described herein may be applied to determining genotypes of duplicated genes or genomic sequences generally.

Congenital Adrenal Hyperplasia (CAH) is a genetic disease involving genome instability caused by genetic duplication. It is characterized by a defect in adrenal steroid biosynthesis causing reduced corticoid production and increased androgen production. CAH manifests in a variety of phenotypic severities, which are broadly classified as Classic and Non-classic disease. Classical disease is more severe, and includes phenotypes such as ambiguous genitalia/sex determination in females in utero (classic virilization), adrenal crisis, and salt wasting. Non-classic disease includes less severe phenotypes including virilization in childhood, and in women, hirsutism, inconsistent menstruation, and infertility. See, e.g., The Merck Manual of Diagnosis and Therapy, 17^{th} ed., Beers and Berkow, Eds. (1999), pages 2381-82.

Approximately 90% of CAH is caused by 21-hydroxylase deficiency attributable to mutations in the gene coding for 21-hydroxylase, also referred to as CYP21A2. The 21-hydroxylase gene lies on chromosome 6p21.3 among genes that code for proteins determining human leukocyte antigen (HLA) types. The 21-hydroxylase gene locus also has a pseudogene called CYP21A, which is about 30 kilobases (kb) away from CYP21A2. CYP21A is approximately 98% homologous in structure to CYP21A2, but is rendered inactive due to minor differences in the gene as compared to the active CYP21A gene. The proximity of CYP21A2 with CYP21A is thought to predispose the CYP21A2 to crossovers in meiosis between CYP21A2 and CYP21A or gene conversion, resulting in loss of gene function.

A number of point mutations and small deletions have been identified that deactivate this second copy. These differences include (in order from exons 1-10): P30L, IVS2-13 A/C>G, Δ8 bp, I172N, exon 6 cluster mutation (I235N, V236E, and M238K), V281L, F306+t, Q318X, and R356W. Transfer of any of these mutations from the pseudogene CYP21A to the functional CYP21A2 can occur through gene conversion or non-homologous recombination, thus inactivating the functional CYP21A2. Another source of mutation at the CYP21 locus is genomic rearrangement resulting in duplication or deletion of entire segments of the gene region. Large 30 kb deletions that fuse CYP21A with CYP21A2 account for approximately 25% of 21-hydroxylase deficiency. These types of mutation at the CYP21 gene locus account for approximately 90% of 21-hydroxylase deficiency. The remaining 10% of 21-hydroxylase deficiency is caused by rare sporadic mutation. The fact that this locus involves an evolutionary gene duplication produces technical difficulties for diagnosing mutation in the functional CYP21A2. Wedell & Luthman, Hum Genet (1993) 91:236-240; Speiser et al., Molecular Genetics and Metabolism, 71: 527-534 (2000); For a review see White & Speiser, Endocrine Reviews 21(3): 245-291 (2000).

The core problem in CAH is the inability of the adrenal glands to make enough cortisol in the non-salt wasting form, or enough cortisol and salt-retaining hormone in the salt-wasting form. Instead of making cortisol, the hormonal raw materials which usually make cortisol are shifted away to make other hormones, specifically male sex hormones (androgens). As a result more androgens are produced than necessary. Before birth, the excess androgens stimulate the growth of the genitalia. When the child is male this does not cause grave problems. However excess androgens in a female with this disorder causes the child's genitalia to have the appearance of a male although the internal genitalia are normal female. The assays of the present invention employ allele-specific amplification to determine a genotype of interest present in a sample of genomic DNA. While the present invention is described in terms of the CYP21 locus, the skilled artisan will understand that the methods described herein are applicable generally to gene loci in which two genes are subject to recombination resulting in variable genotypes present in a population. In various embodiments, the assays comprise four PCR reactions using four primer sequences in various combinations to detect genomic rearrangements at the CYP21 locus, including gene rearrangements and a large 30 kb gene deletion that results in a fusion gene.

In additional preferred embodiments, the assays also utilize primer extension methods in order to further characterize the 21-hydroxylase (CYP21) genotype present in the sample. While the methods are described in terms of the commercially available Applied Biosystems' SNaPshot™ method (Applied Biosystems, Foster City, Calif.), various primer extension methods are known to those of skill in the art. The exemplary methods described herein can detect both wildtype and mutant alleles at nine commonly mutated loci.

The basic strategy of the methods described herein involves amplifying genomic. The resulting amplicon(s) are analyzed by agarose gel electrophoresis to determine which amplicons have been produced. After removing dNTPs and primers from the amplification products, the amplicon(s) are also subjected to a primer extension reaction by combining the PCR products, specific primers, and labeled nucleotides. An enzymatic reaction is then performed and unincorporated ddNTPs are removed. The samples are then analyzed by capillary electrophoresis and the data analyzed. Since each extension primer has a distinct molecular weight, and since each ddNTP has a distinct label (e.g., color) associated with it, the data is analyzed and particular labeled nucleotides are identified (e.g., by size and color) that correlate with the specific genotype present.

By combining information regarding the size and number of amplicons produced, and the size of the extension primer as well as the fluorescent signal from the specific ddNTP that is added in the single base extension reaction, the specific genotype present in the sample is identified.

FIG. 1 provides a schematic illustration of the CYP21 gene region. Referring to FIG. 1 throughout, the active CYP21 gene is called CYP21A2 (or CYP21B in some nomenclature systems), while the duplicated gene (one copy evolutionarily inactivated) is called CYP21A (or the CYP21P pseudogene). If no deletion has occurred, primers 1 and 2 will amplify an approximately 4.0 kb fragment, and primers 3 and 4 will amplify an approximately 3.4 kb fragment. In this case primers 1 and 4 will be physically too far apart to yield a PCR product, and this amplicon will therefore not be detected by agarose gel electrophoresis. Homologous recombination between the active CYP21A2 and the pseudogene CYP21A may result in disease via deletion or gene conversion.

Unequal crossing over between the gene and pseudogene can cause deletion of a 30 kb fragment between the gene and pseudoge and enable production of amplicon with primers 1 and 4. When the deletion has occurred, the genomic sequence complementary to primers 2 and 3 have been removed from the chromosome. An approximately 4.0 kb product will be resolved by agarose electrophoresis.

When a gene rearrangement has occurred producing the reciprocal hybrid gene (A2/A), primers 3 and 2 can now form a PCR product. Thus a PCR product of approximately 3.4 kb will be resolved by agarose gel electrophoresis.

5′ gene conversion (movement of the primer 1 site±other sequence from the pseudogene to the gene ) can also support amplification of the 4 kb product with primers 1 and 4. In this case, primer sites 2 and 3 would not be removed.

Confirmation of allele-specific amplification is provided by digestion with EcoRI. The CYP21A2 active gene contains two EcoRI restriction sites within its coding sequence while the CYP21A pseudogene contains only one EcoRI restriction site. Therefore, referring to FIG. 2, digestion of the 3.4 kb amplicon containing the active CYP21A2 will produce two digestion products, which can be distinguished by agarose gel electrophoresis. Digestion of the 4.0 kb amplicon containing the A/A2 fusion gene will produce three digestion products, while digestion of the 3.4 kb amplicon containing the A2/A rearrangement species will produce only two digestion products.

The CYP21A pseudogene has P30L, IVS2-13A/C>G, Δ8 bp, I172N, exon 6 cluster mutation (I235N, V236E, and M238K), V281L, F306+t, Q318X, and R356W relative to the functional copy. The single base extension (SBE) of oligos that hybridize to the homologous (non-deletion) sequence in the active CYP21A2 are performed and analyzed, preferably with the SNaPshot™ minisequencing kit (Applied Biosystems). This analysis will confirm the presence or absence of the normal (non-mutation) CYP21A2 and/or mutation sequences.

Examples of apparatuses that may be useful for electrophoresis and visualization of amplicons are an agarose gel electrophoresis apparatus, such as CBS Scientific horizontal mini-gel; a power supply having a constant voltage of 100 to 200V or better variable power supply for electrophoresis, such as the BioRad Model 200; photodocumentation apparatus, such as the Alpha Innotech AlphaImager or Polaroid DS34 t; and a transilluminator, e.g., a VWR Model LM-20E or equivalent. Other methods of fractionating the amplification products can also be utilized, such as standard or HPLC chromatography methods, or nucleic acid hybridization microarrays.

Approximately 70-75% of CYP21 disease cases are caused by eight common point mutations, small deletions, insertions, or conversion events. These genotypes can be determined by a number of different methods, all utilizing the 3.4 kb fragment obtained by allele-specific PCR utilizing primers 3 and 4 described above. Preferred primer extension primer locations and sequences are provided in Table 1, along with the genotype they are used to identify.

TABLE 1

| | |
|---|---|
| Primer | |
| Location | Primer Sequence |

| | | | |
|---|---|---|---|
| 1 | P30L | <SEQ ID NO 5> | TTTTCCCGGGGCAAGAGGC |
| 2 | IVS2-13 | <SEQ ID NO 6> | TTTCCAGCTTGTCTGCAGGAGGAG |
| | C>G | | |
| 3 | I172N | <SEQ ID NO 7> | TTTTTTTTTTTCTCCGAAGGTGAG |
| | | | GTAACAG |
| 4 | V281L | <SEQ ID NO 8> | TTTTTTTTTTTTTTTTTGGACAGC |
| | | | TCCTGGAAGGGCAC |
| 5 | Q318X | <SEQ ID NO 9> | TTTTTTTTTTTTTTTTTTTTTTTT |
| | | | TCCCCAGATTCAGCAGCGACTG |
| 6 | R356W | <SEQ ID NO 10> | TTTTTTTTTTTTTTTTTTTTTTTT |
| | | | TTTTTTTTATCGCCGAGGTGCTGC |
| | | | GCCTG |
| 7 | I235N | <SEQ ID NO 11> | TTTTTTTTTTTTTTTTTTTTTTTT |
| | | | TTTTTTTTTTTTTTTTCCATAGAG |
| | | | AAGAGGGAYCACA |
| 8 | V236E | <SEQ ID NO 12> | TTTTTTTTTTTTTTTTTTTTTTTT |
| | | | TTTTTTTTTTTTTTTTTTTTTGCT |
| | | | GCCTCAGCTGCWTCTCC |
| 9 | M238K | <SEQ ID NO 13> | TTTTTTTTTTTTTTTTTTTTTTTT |
| | | | TTTTTTTTTTTTTTTTTTTTTTTT |
| | | | CCTTGTGCTGCCTCAGCTGC |
| 10 | F306+t | <SEQ ID NO 14> | TTTTTTTTTTTTTTTTTTTTTTTT |
| | | | TTTTTTTTTTTTTTTTTTTTTTTT |
| | | | CACCCTCTCCTGGGCCGTGGTTTT |
| | | | TTT |
| 11 | Δ8bp-F | <SEQ ID NO 15> | TTTTTTTTTTTTTTTTTTTTTTTT |
| | | | TTTTTTTTTTTTTTTTTTTTTTTT |
| | | | TTTTTTTTTTTTTTTTACCCGGAC |
| | | | CTGTCSTTG |
| 12 | Δ8bp-R | <SEQ ID NO 16> | TTTTTTTTTTTTTTTTTTTTTTTT |
| | | | TTTTTTTTTTTTTTTTTTTTTTTT |
| | | | TTTTTTTTTTTTTTTTTTGGGCTT |
| | | | TCCAGAGCA |
| 13 | P4535 | <SEQ ID NO 17> | TTTTTTTTTTTTTTTTTTTTTTTT |
| | | | TTTTTTTTTTTTTTTTTTTTTTTT |
| | | | TTTTTTTTTTTTTTTTTTTTTTTT |
| | | | TTTCAGGCCTTCACGCTGCTG |

Numerous detectable labels for incorporation into nucleic acids are known to those of skill in the art. See, e.g., Handbook of Fluorescent Probes and Research Products, 9^{th} ed., Molecular Probes, Inc., 2002, Chapter 8 (“Nucleic Acid Detection and Genomics Technology”). Illustrative fluorescent labels include xanthene dyes, naphthylamine dyes, coumarins, cyanine dyes and metal chelate dyes, such as fluorescein, rhodamine, rosamine, the BODIPY dyes (FL, TMR, and TR), dansyl, lanthanide cryptates, erbium. terbium and ruthenium chelates, e.g. squarates, and the like. Additionally, in certain embodiments, one or more fluorescent moieties can be energy transfer dyes such as those described in Waggoner et al., U.S. Pat. No. 6,008,373. More recently, semiconductor-based quantum dots have also been described for use as biological labels (see, e.g., Bruchez et al., Science 281: 2013-16 (1998)); and mass spectrometry has been described for use in direct detection of unlabeled bases (see, e.g., U.S. Pat. No. 6,043,431)

The oligonucleotides generated by the primer extension methods described herein can be separated from each other so that each is mobilized in a manner that relates to each of their specific sizes. A preferred form of electrophoresis is capillary electrophoresis, but any form of electrophoresis that allows for the separation of a plurality of labeled nucleic acids in small volumes by automated or semi-automated methods and devices may be used. For high throughput of PCR products, an automated capillary electrophoresis (CE) system is used in order to separate labeled DNA molecules in a size-dependent manner, so that signals corresponding to each nucleotide in a sequence are detected in a sequential fashion. For reviews of the use of CE in DNA sequencing and polymorphism analysis, see Heller, Electrophoresis 22:629-43, 2001; Dovichi et al., Methods Mol. Biol. 167:225-39, 2001; Mitchelson, Methods Mol. Biol. 162:3-26, 2001; and Dolnik, J. Biochem. Biophys. Methods 41:103-19, 1999. In the Examples, the ABI PRISM® 3100 Genetic Analyzer may be used with an ABI PRISM 3100 capillary array,36-cm (P/N#4315931). This provides a multi-color fluorescence-based DNA analysis system that uses capillary electrophoresis with 16 capillaries operating in parallel to separate labeled PCR products. A CE DNA sequencer/analyzer that operates 96 capillaries may be preferable in assays wherein 96-well plates are used. Analyzers with the capacity to process 96 wells include the MegaBACE™ 1000 DNA Analysis System (Molecular Dynamics, Inc and Amersham Pharmacia Biotech) and the 3700 DNA Analyzer from (Perkin-Elmer Biosystems)

The following examples serve to illustrate the present invention. These examples are in no way intended to limit the scope of the invention.

### EXAMPLE 1

### Preparation of Genomic DNA

Samples of whole blood were obtained from human subjects, and the blood samples processed to obtain samples of genomic DNA.

Many methods of isolating genomic DNA from blood are known. The following is one example: 10 ml of whole blood is added to 1 ml of proteinase K in a 50 ml tube. 10 ml of Guanidine HCl/Tween solution is added and the sample bump vortexed for 15 seconds. The sample is incubated at 65° C. for 10 min. and centrifuged briefly to remove any drops from the top of the lid. 10 ml of 100% ethanol is then added, the sample vortexed for 15 sec. and then centrifuged briefly to remove any drops from the top of the lid. 20 ml of the mixture is then transferred to the spin filter and centrifuged for 3 min at 2500×g. The remaining mixture is added to the spin filter and centrifuged for 3 min at 2500×g. The spin filter is transferred to a new 50 ml tube making sure ethanol is added to wash concentrate/Guanidine HCl solution before first use. 20 ml of wash concentrate/Guanidine HCl solution is added to the spin filter. The sample is centrifuged for 3 min at 2500×g. The spin filter is removed, the flow through discarded, and the spin filter replaced. 20 ml of wash concentrate/Tris, NaCl is added to the spin filter. The sample is centrifuged for 3 min 2500×g, the-spin filter removed, the flow through discarded, and the spin filter replaced. The sample is centrifuged again for 3 min 2500×g. The spin filter is carefully removed and transferred to a new tube without coming in contact with the wash solution. 10 ml of 10 mM Tris-HCl is added. To increase yields, incubate for 5 min. at 65° C. The tube is centrifuged for 3 min. at 2500×g. The filter unit is removed and the tube lid closed. Genomic DNA in the tube is now ready to use for any application.

In another embodiment the genomic DNA can be isolated by other procedures. For example the KingFisher™ Blood DNA Kit by Thermo LabSystems™ (Vantaa, Finland) provides another commercially available method for isolating genomic DNA. The protocol ensures that only those blood cells containing DNA are isolated for purification. The first step utilizes Pan Leucocyte Dynabeads™ (Dynal Biotech, Oslo, Norway) to isolate the white blood cells. Dynabeads M-450 CD2 are uniform, magnetizable polystyrene beads coated with a primary monoclonal antibody (mAb) specific for the CD2 membrane antigen which is predominantly expressed on human T cells. The mouse IgG1 mAb is attached to Dynabeads™ via a secondary antibody to effect optimal orientation of the primary antibody. Dynabeads™ CD2 are supplied as a suspension containing 4×10⁸ beads/ml in phosphate buffered saline (PBS), pH 7.4, containing 0.1% bovine serum albumin (BSA) and 0.02% sodium azide (NaN₃). In this way, all impurities and unwanted material, such as red blood cells, are left behind. The purified white blood cells are then washed, lysed and mixed with DNA-binding Dynabeads™ which bind the genomic DNA. Subsequent washing steps remove unwanted cell debris and other contaminants, resulting in an average yield of 4-10 μg of highly purified DNA from 200 μl of blood. DNA can be eluted either into microcentrifuge tubes or another suitable vessel.

### EXAMPLE 2

### Duplicated Genes

The following genes are known to be duplicated genes or genes in duplicated regions of the genome. With reference to the present disclosure the person of ordinary skill will realize that these genes will also find use in the present invention. Suitable primers can be designed with reference to commercially available primer design softwares. For example GenomePRIDE is primer design program that designs PCR primers or long oligos on an annotated sequence. GenomePRIDE has been used for the design of DNA arrays/chips containing all ORFs in several organisms. PRIDE is a primer design program that automatically designs primers in single contigs or whole sequencing projects to extend the already known sequence and to double strand single-stranded regions. Both programs are available from Deutsches Krebsforschungszentrum, Technologietransfer (Heidelberg, Germany). Also, Primer Express Sequence Design is available from Applied Biosystems, Foster City, Calif.

C4A/B
HBA1/HBA2
HBB
A2M
ACTGP4/5/6/7/8
Ribosomal RNAs
MRPS18
Transfer RNAs
Pre-rRNAs
28S
5.8S
18S
Cytochrome P450s
(By chromosome)
Chr 1:
CYP4B1
CYP4Z2P
CYP4A11
CYP4A26P
CYP4A27P
CYP4X1
CYP4Z1
CYP4A22
CYP46A4P
Chr 2:
CYP1B1
CYP4F25P
CYP4F32P
CYP26B1
CYP4F31P
CYP27C1
CYP4F27P
CYP4F30P
CYP2C56P
CYP20
CYP27A1
Chr 3:
CYP8B1
CYP51P1
CYP2D31P
Chr 4:
CYP4V2
CYP2U1
CYP4V2
Chr 6:
CYP21A2
CYP21A1P
CYP39A1
CYP2C57P
CYP51P3
Chr 7:
CYP2W1
CYP3A54P
CYP3A55P
CYP3A5
CYP3A51P
CYP3A7
CYP3A4
CYP3A43
CYP3A52P
CYP3A53P
CYP51
CYP5A1
Chr 8:
CYP7A1
CYP11B1
CYP11B2
CYP7B1
Chr 9:
CYP4F26P
CYP4F33P
CYP4F25P
CYP1A8P
Chr 10:
CYP2C61P
CYP26A1
CYP26C1
CYP2C18
CYP2C19
CYP2C58P
CYP2C9
CYP2C59P
CYP2C60P
CYP2C8
CYP17A1
CYP2C62P
CYP2E1
Chr 11:
CYP2R1
Chr 12:
CYP27B1
Chr 13:
CYP434P
CYP51P2
Chr 14:
CYP46
CYP4
Chr 15:
CYP19
CYP1A1
CYP1A2
CYP11A1
Chr 18:
CYP4F35P
Chr 19:
CYP4F22
CYP4F23P
CYP4F8
CYP4F3
CYP4F10P
CYP4F12
CYP4F24P
CYP4F36P
CYP4F2
CYP4F11
CYP4F9P
CYP2T2P
CYP2F1P
CYP2A6
CYP2A7
CYP2G1P
CYP2A18PC
CYP2B7P
CYP2B6
CYP2A18PN
CYP2G2P
CYP2F1
CYP2T3P
CYP2S1
Chr 20:
CYP28A1
CYP24
Chr 21:
CYP2C63P
CYP4F29P
Chr 22:
CYP2D8P
CYP2D7AP
CYP2D6
Chr X:
CYP2C64P

### EXAMPLE 3

### Preparation of Master Mixes For CAH Analysis

Each master mix provides the primers necessary for carrying out the four PCRs that will determine the result of the assay. The master mixes are ed in the PCR procedure were prepared as follows:

Master Mix #1 (CYP21A2 Amplification)

| Components | for 1 Rxn | for 110 Rxns |
|---|---|---|
| H₂O | 29.6 μL | 3256 μL |
| 10x Qiagen ™ PCR buffer | 5.5 μL | 605 μL |
| 5x Q solution | 10.0 μL | 1100 μL |
| 25 mM dNTP mix | 0.5 μL | 55 μL |
| 10 μM primer 3 | 1.5 μL | 165 μL |
| 10 μM primer 4 | 1.5 μL | 165 μL |
| 5 μM AVPR2 primer mix* | 1.5 μL | 165 μL |
| Total | 50.0 μL | 5511 μL |

Master Mix #2 (CYP21A Amplification)

| Components | for 1 Rxn | for 110 Rxns |
|---|---|---|
| H₂O | 29.6 μL | 3256 μL |
| 10x Qiagen ™ PCR buffer | 5.5 μL | 605 μL |
| 5x Q solution | 10.0 μL | 1100 μL |
| 25 mM dNTP mix | 0.5 μL | 55 μL |
| 10 μM primer 1 | 1.5 μL | 165 μL |
| 10 μM primer 2 | 1.5 μL | 165 μL |
| 5 μM AVPR2 primer mix* | 1.5 μL | 165 μL |
| Total | 50.0 μL | 5511 μL |

Master Mix #3 (30 kb Deletion Product Amplification)

| Components | for 1 Rxn | for 110 Rxns |
|---|---|---|
| H₂O | 29.6 μL | 3256 μL |
| 10x Qiagen ™ PCR buffer | 5.5 μL | 605 μL |
| 5x Q solution | 10.0 μL | 1100 μL |
| 25 mM dNTP mix | 0.5 μL | 55 μL |
| 10 μM primer 1 | 1.5 μL | 165 μL |
| 10 μM primer 4 | 1.5 μL | 165 μL |
| 5 μM AVPR2 primer mix* | 1.5 μL | 165 μL |
| Total | 50.0 μL | 5511 μL |

Master Mix #4 (Genomic Rearrangement Product Amplification)

| Components | for 1 Rxn | for 110 Rxns |
|---|---|---|
| H₂O | 29.6 μL | 3256 μL |
| 10x Qiagen ™ PCR buffer | 5.5 μL | 605 μL |
| 5x Q solution | 10.0 μL | 1100 μL |
| 25 mM dNTP mix | 0.5 μL | 55 μL |
| 10 μM primer 3 | 1.5 μL | 165 μL |
| 10 μM primer 2 | 1.5 μL | 165 μL |
| 5 μM AVPR2 primer mix* | 1.5 μL | 165 μL |
| Total | 50.0 μL | 5511 μL |

*AVPR2 primers are AVPR2F: <SEQ ID NO 18> TGACCATCCCTCTCAATCTTC; and AVPR2R: <SEQ ID NO 19> TCCCTCTTTCCTGCCACTCCT.

A sufficient quantity of Taq polymerase was added to master mix tubes. While any suitable Taq polymerase can be used, in a preferred embodiment HotStarTaq™ is utilized (Qiagen, Valencia, Calif.). HotStarTaq™ is a modified form of Taq DNA Polymerase. It is supplied in an inactive state that has no polymerase activity at ambient temperatures. This prevents extension of nonspecifically annealed primers and primer-dimers formed at low temperatures during PCR setup and the initial PCR cycle. HotStarTaq™ DNA Polymerase is activated by a 15-minute incubation at 95° C. which can be incorporated into any existing thermal-cycler program.

HotStarTaq™ Master Mix is a premixed solution containing HotStarTaq™ DNA Polymerase, PCR Buffer, and dNTPs. The solution provides a final concentration of 1.5 mM MgCl₂ and 200 μM of each dNTP. The PCR buffer contains a combination of KCl and (NH₄)₂SO₄ in the buffer to promote specific primer-template annealing and reduce nonspecific annealing, thereby maximizing yields of specific PCR product

A PCR additive is also used in the most preferred embodiments to assist in extending PCR. The preferred PCR additive is Taq Extender™ PCR additive, which improves the reliability and yield of conventional Taq-based PCR amplifications. Taq Extender™ additive increases the efficiency of Taq DNA polymerase extension reactions during each cycle of PCR by reducing mismatch pausing, thus resulting in a greater percentage of completed extension reactions. In addition, Taq Extender™ PCR additive improves the PCR amplification of difficult templates and increases the reliability and yield of many PCR targets up to 35 kb in length. The additive is simply added to amplification reactions in an amount equal to that of Taq DNA polymerase, the standard buffer is replaced with an optimized 10× Taq Extender™ buffer and standard PCR cycling conditions are used. Taq Extender™ buffer contains 200 mM Tris-HCl (pH 8.8), 100 mM KCl, 100 mM (NH₄)₂SO₄, 20 mM MgSO₄, 1% Triton X-100, and 1 mg/ml nuclease-free bovine serum albumin. Nielson, K. B., et al. (1994) Strategies 7: 27; Innis et al., (1988) Proc Natl Acad Sci USA, 85(24): 9436-40.

The person of ordinary skill will realize that many other Taq polymerases are commercially available or that can be manufactured. For example, another embodiment utilizes SureStart™ Taq DNA polymerase, which is commercially available (Stratagene, La Jolla, Calif.). The enzyme can also be used effectively in amplification reactions to achieve reduced background and high yield of PCR product. The enzyme consists of a reversibly inactivated DNA polymerase that remains inactive until stringent denaturation temperatures are reached. It is activated by adding a heat step (9 to 12 minutes at 92 to 95° C.) to the beginning of thermal cycling programs (pre-PCR heat-activation method). Alternatively, the pre-PCR heat-activation step can be omitted to permit enzyme activation during temperature cycling. When this method is employed, it is preferable to add additional cycles to existing cycling programs to achieve optimal product yield.

### EXAMPLE 4

### PCR Amplification for CAH Analysis

A template is created showing the position of each sample/control to be amplified and identifying which samples receive which Master Mix and other ingredients.

For each sample for PCR reactions were set up. A Master Mix, HotStarTaq™, Taq Extender™, and DNA-were added to the wells of a 96 well plate. The plate was vortexed (without creating bubbles) and transferred to a thermal cycler (Perkin-Elmer GeneAmp 9700, Perkin-Elmer Instruments, Boston, Mass.). The reaction was then started. The program for the thermal cycler was as follows: Denature 94° C. 10 sec, Anneal 55° C. 1 min, Elongate 68° C. 3:42 min, repeat for 10 cycles. Then a program was followed according to the following: Denature 94 C° for 10 sec., Anneal 55° C. for 1 min, Elongate 68° C. for 3:42 min, repeat for 30 cycles and increase the elongation by 10 seconds with each cycle. Finally, the sample was held at 68° C. for 5 min, and then held overnight at 10° C.

Following PCR, Shrimp Alkaline Phosphatase (SAP) and Exonuclease I (Exo I) are preferably used to prepare PCR products for base extension. SAP removes the phosphate groups from the excess dNTPs left over from the PCR reaction, and Exonuclease I digests the single stranded PCR primers into dNTPs. Both enzymes are readily inactivated by heating at 75° C. for 15 min and therefore do not affect the single base extension reaction. 2 unit of SAP and 1 units of ExoI were added to 1×SAP buffer to a final volume of 6 μl per reaction. The parameters for the Exo/SAP reaction were 37° C. for 2 hours, 75° C. for 15 minutes, and hold at 4° C. SAP buffer contains 25 mM Tris-HCl, 10 mM MgCl₂, (pH 9.0 at 37° C.).

### EXAMPLE 5

### Agarose Electrophoresis for CAH Analysis

The PCR amplicons were assayed on a 0.7% agarose gel. Amplification was confirmed by the presence of a 2 kb internal control band. Approximate sizes of amplicons as compared to the size of the standard are:

CYP21A2=3.4 kb

CYP21A=4.0 kb

Hybrid 1=4.0 kb (30 kb deletion product of homologous recombination or gene conversion without deletion—A/A2)

Hybrid 2=3.4 kb (reciprocal rearranged gene—A2/A)

Internal Control=2.0 kb

5 μl of TE buffer (0.01 M Tris, 0.05 M EDTA, pH 7.5) was added to 5 μl of PCR product. For the standard lane, 1 μl of 1 kb ladder and 10 μl of TE was combined. 2 μl of 6× loading buffer (Blue/Orange 6× loading dye (Promega, Madison, Wis.)) was added to all wells and the sample electrophoresed at 100 V for about 2 hours. Those samples showing positive PCR amplicons were transferred to a fresh PCR tray for “clean-up” of the PCR amplicons. 30 μl of SAP/ExoI was added to each well and incubations performed, again according to this following program: 37 C for 2 hours; 75 C for 15 min; hold at 4 C.

### EXAMPLE 6

### Single Base Extension for CAH Analysis

Primer extension mix was prepared by dispensing the various primers into a tube. The primers included extension primers listed in Table 1 and were prepared according to Table 2:

TABLE 2

| | | | Volume | |
|---|---|---|---|---|
| | | | primer for | Volume |
| | Primer Stock | Primer Final | full plate | primer for |
| Primer | Concentration | Concentration | (115 Rxns, | large scale |
| Location | (μM) | (μM) | μL) | storage (μL) |
| P30L | 100 | 4 | 4.6 | 80 |
| I2GIVS2- | 100 | 8 | 9.2 | 160 |
| I172N | 100 | 6 | 6.9 | 120 |
| V281L | 100 | 2 | 2.3 | 40 |
| Q318X | 100 | 8 | 9.2 | 160 |
| R356W | 100 | 8 | 9.2 | 160 |
| I235N | 100 | 6 | 6.9 | 120 |
| V236E | 100 | 10 | 11.5 | 200 |
| M238K | 100 | 10 | 11.5 | 200 |
| F306+t | 100 | 2 | 2.3 | 40 |
| Δ8bp-F | 100 | 6 | 6.9 | 120 |
| Δ8bp-R | 100 | 6 | 6.9 | 120 |
| P453S | 100 | 8 | 9.2 | 160 |
| H2O | | | 18.4 | 320 |
| Total | | | 115 μL | 2000 μL |

5 μl of reaction mix containing the distinctively labeled ddNTPs and 1 unit of DNA polymerase in a 20 μl reaction volume of buffer (10 mM KCl, 20 mM Tris-HCl (pH 8.8 at 25 C), 10 mM (NH₄)₂SO₄, 2 mM MgSO₄ and 0.1% Triton X-100), 5 μl of SAP/ExoI-treated PCR product, and 1 μl of primer extension mix was added to a fresh 96 well tray (or in strip cap 0.2 μl tubes). The plate was sealed and vortexed to mix, with a quick spin to collect samples. The plate was transferred to the thermal cycler and subjected to the following parameters for single base extension: Denature 96° C. for 30 seconds, Anneal 50° C. for 5 seconds, Elongate at 60° C. for 30 seconds, repeat for 25 cycles, hold at 10° C. overnight.

A second SAP reaction was then performed by adding 1 μl of SAP to each well. The plate was sealed, vortexed to mix, and quick spun to collect samples. The plate was then transferred back to the thermal cycler and subjected to the following parameters: 37° C. for 1 hour, 75° C. for 15 minutes, and hold at 4° C.

### EXAMPLE 7

### Capillary Electrophoresis and Detection for CAH Analysis

Capillary electrophoresis is an analytical technique that separates species by applying voltage across buffer filled capillaries. It is generally used for separating ions, which move at different speeds when the voltage is applied depending on their size and charge. The solutes are seen as peaks as they pass through the detector and the area of each peak is proportional to their concentration, which allows quantitative determinations. Analysis times are in the region of 1-30 minutes depending on the complexity of the separation. Modem instruments are relatively sophisticated and may contain fibre optical detection systems, high capacity autosamplers, and temperature control devices. Detection is usually by UV absorbance, often with a diode array. Fluorescence detection is the preferred mode of detection. Indirect UV detection is widely used for detecting solutes having no chromophores such as metal ions or inorganic anions. Low UV wavelengths (e.g., 190-200 nm) are also used to detect simple compounds such as organic acids.

An ABI PRISM® 310 Genetic Analyzer (Applied Biosystems, Foster City, Calif.) was used for the capillary electrophoresis analysis. The ABI PRISM® system is a fluorescence-based, fully automated DNA analysis system using capillary electrophoresis with 16 capillaries operating in parallel. Samples were prepared by mixing 1 μl of each sample with 20 μl of Hi-Di™ formamide (Applied Biosystems, Foster City, Calif.) and 1 μl of LIZ™ internal size standard (Applied Biosystems) in a 96-well tray or in strip cap 0.2 μl tubes. The wells were covered and the plate vortexed to mix. The plate was then heated at 92-95° C. for 3-5 minutes in a heat block, and then transferred to ice. The ABI PRISM® was set up and operated according to the manufacturer's instructions.

The SNaPshot™ Multiplex System consists of the SNaPshot™ Multiplex Kit, together with GeneScan™ 120 LIZ™Size Standard, Genotyper® Software version 3.7, and the ABI PRISM® capillary electrophoresis instrument (all from Applied Biosystems). This system is commercially available and is a preferred embodiment for high-throughput SNP validation and medium-throughput linkage and association studies. Thousands of SNPs per day can be determined using this system.

The assay system allows multiplexing during single base extension (SBE) of at least 13 primer-template combinations in a single tube/single capillary format. Therefore it is highly preferred for the determination of which nucleotide is added in the present application. Separation of SNP loci can be determined down to one base-pair. In the single base extension reaction primers that bind to particular nucleic acid sequences are extended by a single nucleotide. The ddNTPs are provided in the reaction, and each type of ddNTP is provided with a distinct fluorescent label in the preferred embodiment.

Therefore, it is determined that the combination of the molecular weight differences in the extension primers (due to the poly-T tail) and the fluorescent labeling of the single base extension reactions enables the precise identification of the genotype at the CYP21 gene. Specific primers have been correlated with specific genotypes. Because those primers have been engineered as described herein to have different molecular weights (conferred by the differing poly-T tails attached to them), one level of identification is obtained by identification of the presence of a labeled primer. Through the single base extension reaction the identity of particular nucleotides is also determined, thereby enabling the user to definitively identify the primers present and specifically match those primers to particular genotypes. Determination of the genotypes is preferably done in an automated fashion using a suitable software, such as the Genotyper® software. Genomic rearrangements and specific genotypes were identified according to Table 3 below:

TABLE 3

| | Peak Size | | |
|---|---|---|---|
| Allele | (bp) | wt | Mutant |
| P30L | 22-28 | blue | green |
| IVS2-13 | 29-33 | red/blue | black |
| A/C > G | | | |
| I172N | 34-36 | green | red |
| V281L | 39-42 | blue | red |
| Q318X | 45-50 | black | red |
| R356W | 54-58 | black | red |
| I235N | 62-64 | red | green |
| V236E | 67-69 | green | red |
| M238K | 69-71 | green | red |
| F306+t | 78-80 | blue | red |
| Δ8bp-F | 84-87 | blue | red |
| Δ8bp-R | 88-90 | blue | black |
| P453S | 94-96 | black | red |

While the techniques describe above are the most preferred embodiment, in other embodiments other techniques can be used to determine the genotypes. In one embodiment the Taqman™ Allelic Discrimination system (Applied Biosystems, Foster City, Calif.) can be used instead of the SnaPshot™ system. TaqMan® Pre-Developed Assay Reagents for Allelic Discrimination (PDARs for AD)are available for the ABI PRISM® 7700 (Applied Biosystems) and the ABI PRISM® 7900 HT Sequence Detection Systems (Applied Biosystems). The TaqMan® PDARs for AD use the 5′ nuclease assay to genotype purified DNA samples for specific mutations. Most TaqMan® PDARs for AD assays discriminate between two alleles of single nucleotide polymorphisms (SNPs). Each assay contains two different TaqMan® probes, and each uniquely labeled probe binds preferentially to one of the alleles.

The TaqMan® probes contain two additional features that provide better allelic discrimination. The minor groove binder (MGB) enhances the discrimination between match and mismatch by allowing the use of shorter probes. Second, a non-fluorescent quencher allows better spectral discrimination between the reporter dyes.

TaqMan® PDARs for AD are convenient to use because reaction set-up involves only four components including the sample. Since genotyping is done by endpoint reading the TaqMan® PDAR reaction, high-volume users can use multiple thermal cyclers to amplify DNA before the cycled reactions are analyzed.

In still more embodiments other methods of genotyping the mutants can be used. As an alternative to the methods described above, primer extension with fluorescently labelled ddNTPs can also be performed on widely available automated sequencers. One variation of the methods above uses extension primers labelled with 3 different dyes (FAM, HEX, and TET). Size standards can be designed ranging from 13 to 43 bp, labelled with TAMRA. In one embodiment, the 3′ end of the primer extends not immediately up to the SNP, but one bp earlier. This reduces the interference of the first extension peak with the peak produced by the primer which has not incorporated any d/ddNTP. Three dNTPs and 1 ddNTP can be used for each reaction, so that the primer extends between 1 and 5 bp beyond the SNP, until it reaches the next ddNTP site. This produces 3 peaks which allow unambiguous genotype determination. The presence of a peak produced by the non-extended primer in each reaction allows failed reactions to be distinguished and provides an internal size control. Using different sizes of primers, ranging from 15 to 31 bp, 8 SNPs can be multiplexed for analysis on the same gel at low cost.

The invention illustratively described herein may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

Other embodiments are set forth within the following claims.

1. A method of determining the genotype of a duplicated genomic sequence comprising a first distinct genomic sequence and a second distinct genomic sequence, wherein said first and second distinct genomic sequences are predisposed to recombination or genetic exchange to form a hybrid gene, the method comprising:
(a) amplifying a DNA sample using amplification primers configured and arranged to produce distinguishable amplicons from each of said first distinct genomic sequence, second distinct genomic sequence, and one or more hybrid genes; and
(b) detecting the amplicons produced in step (a), whereby said genotype is determined.

2. A method of determining the genotype of a duplicated gene comprising a first distinct gene and a second distinct gene, wherein said first and second distinct genes are predisposed to recombination to form hybrid genes, the method comprising:
(a) amplifying a DNA sample using four amplification primers to produce
(i) a first amplicon corresponding to the first distinct gene, utilizing a first primer and a second primer selected to produce the first amplicon in the presence of the first distinct gene, but not in absence of the first distinct gene,
(ii) a second amplicon corresponding to the second distinct gene, utilizing a third primer and a fourth primer selected to produce the second amplicon in the presence of the second distinct gene, but not in the absence of the second distinct gene,
(iii) a third amplicon corresponding to a hybrid gene, if present, that comprises a portion of the first distinct gene and a portion of the second distinct gene utilizing one of said first primer and second primer and one of said third primer and fourth primer to produce the third amplicon in the presence of the hybrid gene, but not in absence of the hybrid gene, and
(b) determining which of the first, second, and third amplicons are produced, to determine the genotype at the duplicated region of the chromosome in the sample.

3. The method of claim 2 wherein one of said first and second distinct genes is a functional gene, and the other of said first and second distinct genes is a pseudogene.

4. The method of claim 2 wherein the third amplicon corresponds to a fusion gene of the first distinct gene and the second distinct gene.

5. The method of claim 2 wherein the third amplicon corresponds to a rearranged gene of the first distinct gene and the second distinct gene.

6. The method of claim 2 wherein said four amplification primers are selected to distinguish between a fusion gene of the first distinct gene and the second distinct gene and a rearranged gene of the first distinct gene and the second distinct gene.

7. The method of claim 2, further comprising determining the presence or absence of one or more single nucleotide polymorphisms in one or more of said amplicons.

8. The method of claim 7, wherein the step of determining the presence or absence of one or more single nucleotide polymorphisms comprises contacting one or more of the resulting amplicon(s) with one or more extension primers under conditions where the extension primers are extended by the addition of a distinctively labeled ddNTP from a set of ddNTPs;
determining which distinctively labeled ddNTP is present in each of the extended extension primer(s); and
correlating the presence of distinctively labeled ddNTPs with a specific genotype at the duplicated region of the chromosome.

9. The method of claim 8 wherein each of the extension primers comprises a different molecular weight due to the presence of a unique number of nucleotide residues in each extension primer.

10. The method of claim 8 wherein the ddNTPs comprise a fluorescent label.

11. The method of claim 8 wherein the set of ddNTPs comprise ddATP, ddCTP, ddGTP, and ddTTP, each of which is labeled with a distinct fluorescent label.

12. The method of claim 2 wherein the method further comprises separation of the amplicon(s) produced by agarose gel electrophoresis.

13. The method of claim 8 wherein the method further comprises separation of the extended extension primer(s) by capillary electrophoresis.

14. The method of claim 2 wherein the presence or absence of any of the amplicons is associated with a disease.

15. The method of claim 14 wherein the disease is selected from the group consisting of Gaucher disease, familial juvenile nephronophthisis, fascioscapulohumeral muscular dystrophy, spinal muscular atrophy, polycystic kidney disease, Chronic Granulomatous disease, Hunter syndrome, Charcot-Marie tooth disease (CMT1A)/Hereditary neuropathy with liability to pressure palsies, Neurofibromatosis, Ichthyosis, Red-green color blindness, Hemophilia A, Incontinentia pigmenti, Emery Dreifuss muscular dystrophy, Shwachman-Diamond Syndrome, Williams-Beuren syndrome, Angelman syndrome/15q11.2q13 Prader-Willi syndrome, DiGeorge/Velocardiofacial syndrome, debrisoquine, Chr 22-BL, Chr 22HsPOX2/DGR6 Schizophrenia, Smith-Magenis syndrome, Dup 17p11.2 syndrome, Cat Eye Syndrome, Chr 22-BE1, Chr 22-BE2, and Chr 22-BK.

16. The method of claim 2, further comprising amplifying said DNA sample using two or more additional amplification primers selected to amplify one or more control nucleic acid sequences

17. A method of determining a CYP21 genotype in a sample comprising DNA, comprising,
(a) amplifying the DNA using four amplification primers to produce
(i) a first amplicon corresponding to a full length CYP21A gene if present, utilizing a first primer and a second primer selected to produce the first amplicon in the presence of the full length CYP21A gene, but not in absence of the full length CYP21A gene,
(ii) a second amplicon corresponding to a full length CYP21A2 gene if present, utilizing a third primer and a fourth primer selected to produce the second amplicon in the presence of the full length CYP21A2 gene, but not in the absence of the full length CYP21A2 gene,
(iii) a third amplicon corresponding to a CYP21A/CYP21A2 fusion gene if present utilizing one of said first primer and second primer and one of said third primer and fourth primer to produce the third amplicon in the presence of the CYP21A/CYP21A2 fusion gene, but not in absence of the CYP21A/CYP21A2 fusion gene, and
(iv) a fourth amplicon corresponding to a CYP21A2/CYP21A rearranged gene if present utilizing one of said first primer and second primer and one of said third primer and fourth primer to produce the fourth amplicon in the presence of the CYP21A2/CYP21A rearranged gene, but not in absence of the CYP21A2/CYP21A rearranged gene; and
(b) determining which of the first, second, third, and fourth amplicons are produced, to determine the CYP21A2 genotype in the sample.

18. The method of claim 17, further comprising contacting one or more of the resulting amplicon(s) with one or more extension primers under conditions where the extension primers are extended by the addition of a distinctively labeled ddNTP from a set of ddNTPs; and
determining which distinctively labeled ddNTP is present in each of the extended extension primer(s).

19. The method of claim 18 wherein each of the extension primers comprises a different molecular weight due to the presence of a unique number of nucleotide residues in each extension primer.

20. The method of claim 19 wherein the different molecular weights are due to the presence of nucleotide residues that do not hybridize to the CYP21A or CYP21A2 genes.

21. The method of claim 20 wherein the nucleotide residues that do not hybridize to the CYP21A or CYP21A2 genes comprise a plurality of thymidine nucleotide residues.

22. The method of claim 18 wherein the ddNTPs comprise a fluorescent label.

23. The method of claim 22 wherein the set of ddNTPs comprise ddATP, ddCTP, ddGTP, and ddTTP, each of which is labeled with a distinct fluorescent label.

24. The method of claim 17 wherein
the first amplicon is a PCR product of about 4.0 kb in length;
the second amplicon is a PCR product of about 3.4 kb in length;
the third amplicon is a PCR product of about 4.0 kb in length; and
the fourth amplicon is a PCR product of about 3.4 kb in length.

25. The method of claim 16 wherein
the first and second primers flank the CYP21A2 gene if present; and
the third and fourth primers flank the CYP21A gene if present;
the third and second primers flank the CYP21A2/CYP21A rearranged gene if present; and
the first and fourth primers flank the CYP21A/CYP21A2 fusion gene if present.

26. The method of claim 17 wherein one or more of said first, second, third, or fourth primers are selected from the group consisting of:

| | |
|---|---|
| <SEQ ID NO 1> | TCCCCAATCCTTACTTTTTGTC; |
| <SEQ ID NO 2> | CCTCAATCCTCTGCGGCA; |
| <SEQ ID NO 3> | GCTTCTTGATGGGTGATCAAT; |
| and | |
| <SEQ ID NO 4> | CCTCAATCCTCTGCAGCG; |

or complementary sequences thereof.

27. The method of claim 22 wherein
the first primer has the sequence <SEQ ID NO 1> TCCCCAATCCTTACTTTTTGTC;
the second primer has the sequence <SEQ ID NO 2> CCTCAATCCTCTGCGGCA;
the third primer has the sequence <SEQ ID NO 3> GCTTCTTGATGGGTGATCAAT; and
the fourth primer has the sequence <SEQ ID NO 4> CCTCAATCCTCTGCAGCG.

28. The method of claim 17 wherein the method further comprises separation of the amplicon(s) produced by agarose gel electrophoresis.

29. The method of claim 18 wherein the method further comprises separation of the extended extension primer(s) by capillary electrophoresis.

30. The method of claim 18 wherein the extension primers bind to one or more sequences characteristic of a genotype selected from the group consisting of: IVS2-13 A/C>G, I172N, V281L, Q318X, R356W, 1235N, V236E, M238K, F306+t, Δ8 bp-R, P30L, Δ8 bp-F, and P453S.

31. The method of claim 17, further comprising amplifying said DNA sample using two or more additional amplification primers selected to amplify one or more control nucleic acid sequences.

32. An aqueous solution comprising one or more nucleic acids having sequences selected from the group consisting of:

| | |
|---|---|
| <SEQ ID NO 1> | TCCCCAATCCTTACTTTTTGTC; |
| <SEQ ID NO 2> | CCTCAATCCTCTGCGGCA; |
| <SEQ ID NO 3> | GCTTCTTGATGGGTGATCAAT; |
| and | |
| <SEQ ID NO 4> | CCTCAATCCTCTGCAGCG; |

or a complementary or conservative nucleic acid sequence thereof.

33. An aqueous solution comprising each of the nucleic acids having sequences selected from the group consisting of:

| | |
|---|---|
| <SEQ ID NO 5> | TTTTCCCGGGGCAAGAGGC; |
| <SEQ ID NO 6> | TTTCCAGCTTGTCTGCAGGAGGAG; |
| <SEQ ID NO 7> | TTTTTTTTTTTCTCCGAAGGTGAGGTAACAG; |
| <SEQ ID NO 8> | TTTTTTTTTTTTTTTTTGGACAGCTCCTGGAAGG |
| | GCAC; |
| <SEQ ID NO 9> | TTTTTTTTTTTTTTTTTTTTTTTTTCCCCAGATT |
| | CAGCAGCGACTG; |
| <SEQ ID NO 10> | TTTTTTTTTTTTTTTTTTTTTTTTTTTTTTTTAT |
| | CGCCGAGGTGCTGCGCCTG; |
| <SEQ ID NO 11:> | TTTTTTTTTTTTTTTTTTTTTTTTTTTTTTTTTT |
| | TTTTTTCCATAGAGAAGAGGGAYCACA; |
| <SEQ ID NO 12: | TTTTTTTTTTTTTTTTTTTTTTTT TTTTTTTTT |
| | TTTTTTTTTTTTGCTGCCTCAGCTGCWTCTCC; |
| <SEQ ID NO: 13> | TTTTTTTTTTTTTTTTTTTTTTTTTTTTTTTTTT |
| | TTTTTTTTTTTTTTCCTTGTGCTGCCTCAGCT |
| | GC; |
| <SEQ ID NO: 14> | TTTTTTTTTTTTTTTTTTTTTTTTTTTTTTTTTT |
| | TTTTTTTTTTTTTTCACCCTCTCCTGGGCCGTGG |
| | TTTTTTT; |
| <SEQ ID NO: 15> | TTTTTTTTTTTTTTTTTTTTTTTTTTTTTTTTTT |
| | TTTTTTTTTTTTTTTTTTTTTTTTTTTTTTACCC |
| | GGACCTGTCSTTG; |
| <SEQ ID NO 16> | TTTTTTTTTTTTTTTTTTTTTTTTTTTTTTTTTT |
| | TTTTTTTTTTTTTTTTTTT TTTTTTTTTTTTTG |
| | GGCTTTCCAGAGCA; |
| <SEQ ID NO 17> | TTTTTTTTTTTTTTTTTTTTTTTTTTTTTTTTTT |
| | TTTTTTTTTTTTTTTTTTTTTTTTTTTTTTTTTT |
| | TTTTTTTCAGGCCTTCACGCTGCTG; |

or a complementary or conservative nucleic acid sequence thereof.

34. The method of claim 18 wherein one or more extension primers are selected from the group consisting of:

| | |
|---|---|
| <SEQ ID NO 5> | TTTTCCCGGGGCAAGAGGC; |
| <SEQ ID NO 6> | TTTCCAGCTTGTCTGCAGGAGGAG; |
| <SEQ ID NO 7> | TTTTTTTTTTTCTCCGAAGGTGAGGTAACAG; |
| <SEQ ID NO 8> | TTTTTTTTTTTTTTTTTGGACAGCTCCTGGAAGG |
| | GCAC; |
| <SEQ ID NO 9> | TTTTTTTTTTTTTTTTTTTTTTTTTCCCCAGATT |
| | CAGCAGCGACTG; |
| <SEQ ID NO 10> | TTTTTTTTTTTTTTTTTTTTTTTTTTTTTTTTAT |
| | CGCCGAGGTGCTGCGCCTG; |
| <SEQ ID NO 11:> | TTTTTTTTTTTTTTTTTTTTTTTTTTTTTTTTTT |
| | TTTTTTCCATAGAGAAGAGGGAYCACA; |
| <SEQ ID NO 12: | TTTTTTTTTTTTTTTTTTTTTTTT TTTTTTTTT |
| | TTTTTTTTTTTTGCTGCCTCAGCTGCWTCTCC; |
| <SEQ ID NO: 13> | TTTTTTTTTTTTTTTTTTTTTTTTTTTTTTTTTT |
| | TTTTTTTTTTTTTTCCTTGTGCTGCCTCAGCT |
| | GC; |
| <SEQ ID NO: 14> | TTTTTTTTTTTTTTTTTTTTTTTTTTTTTTTTTT |
| | TTTTTTTTTTTTTTCACCCTCTCCTGGGCCGTGG |
| | TTTTTTT; |
| <SEQ ID NO: 15> | TTTTTTTTTTTTTTTTTTTTTTTTTTTTTTTTTT |
| | TTTTTTTTTTTTTTTTTTTTTTTTTTTTTTACCC |
| | GGACCTGTCSTTG; |
| <SEQ ID NO 16> | TTTTTTTTTTTTTTTTTTTTTTTTTTTTTTTTTT |
| | TTTTTTTTTTTTTTTTTTT TTTTTTTTTTTTTG |
| | GGCTTTCCAGAGCA; |
| <SEQ ID NO 17> | TTTTTTTTTTTTTTTTTTTTTTTTTTTTTTTTTT |
| | TTTTTTTTTTTTTTTTTTTTTTTTTTTTTTTTTT |
| | TTTTTTTCAGGCCTTCACGCTGCTG; |

35. A method of diagnosing congenital adrenal hyperlasia in a patient, comprising:
(a) amplifying a sample of DNA from the patient to produce
(i) a first amplicon corresponding to a full length CYP21A gene if present, utilizing a first primer and a second primer selected to produce the first amplicon in the presence of the full length CYP21A gene, but not in absence of the full length CYP21A gene,
(ii) a second amplicon corresponding to a full length CYP21A2 gene if present, utilizing a third primer and a fourth primer selected to produce the second amplicon in the presence of the full length CYP21A2 gene, but not in the absence of the full length CYP21A2 gene,
(iii) a third amplicon corresponding to a CYP21A/CYP21A2 fusion gene if present utilizing one of said first primer and second primer and one of said third primer and fourth primer to produce the third amplicon in the presence of the CYP21A/CYP21A2 fusion gene, but not in absence of the CYP21A/CYP21A2 fusion gene, and
(iv) a fourth amplicon corresponding to a CYP21A2/CYP21A rearranged gene if present utilizing one of said first primer and second primer and one of said third primer and fourth primer to produce the fourth amplicon in the presence of the CYP21A2/CYP21A rearranged gene, but not in absence of the CYP21A2/CYP21A rearranged gene;
(b) contacting one or more of the resulting amplicon(s) with one or more extension primers under conditions where the extension primers are extended by the addition of a distinctively labeled ddNTP from a set of ddNTPs; and
(c) determining which of the first, second, third, and fourth amplicons are produced, and which distinctively labeled ddNTP is present in each of the extended extension primer(s) to diagnose congenital adrenal hyperplasia in the patient.

36. A kit comprising one or more nucleic acids selected from the group consisting of:

| | |
|---|---|
| <SEQ ID NO 1> | TCCCCAATCCTTACTTTTTGTC; |
| <SEQ ID NO 2> | CCTCAATCCTCTGCGGCA; |
| <SEQ ID NO 3> | GCTTCTTGATGGGTGATCAAT; |
| and | |
| <SEQ ID NO 4> | CCTCAATCCTCTGCAGCG; |

or complementary or conservative nucleic acid sequences thereof, in an amount sufficient to perform a polymerase chain reaction amplification of a nucleic acid sample.

37. A kit of claim 36 wherein the one or more nucleic acids are each of

| | |
|---|---|
| <SEQ ID NO 1> | TCCCCAATCCTTACTTTTTGTC; |
| <SEQ ID NO 2> | CCTCAATCCTCTGCGGCA; |
| <SEQ ID NO 3> | GCTTCTTGATGGGTGATCAAT; |
| and | |
| <SEQ ID NO 4> | CCTCAATCCTCTGCAGCG; |

in an amount sufficient to perform a polymerase chain reaction amplification of a nucleic acid sample.

38. A kit of claim 36 further comprising reagents for performing a single base extension reaction, wherein the nucleic acids and reagents are provided in a container.

39. A kit of claim 38 comprising each of the nucleic acids selected from the group consisting of:

| | |
|---|---|
| <SEQ ID NO 5> | TTTTCCCGGGGCAAGAGGC; |
| <SEQ ID NO 6> | TTTCCAGCTTGTCTGCAGGAGGAG; |
| <SEQ ID NO 7> | TTTTTTTTTTTCTCCGAAGGTGAGGTAACAG; |
| <SEQ ID NO 8> | TTTTTTTTTTTTTTTTTGGACAGCTCCTGGAAGG |
| | GCAC; |
| <SEQ ID NO 9> | TTTTTTTTTTTTTTTTTTTTTTTTTCCCCAGATT |
| | CAGCAGCGACTG; |
| <SEQ ID NO 10> | TTTTTTTTTTTTTTTTTTTTTTTTTTTTTTTTAT |
| | CGCCGAGGTGCTGCGCCTG; |
| <SEQ ID NO 11:> | TTTTTTTTTTTTTTTTTTTTTTTTTTTTTTTTTT |
| | TTTTTTCCATAGAGAAGAGGGAYCACA; |
| <SEQ ID NO 12: | TTTTTTTTTTTTTTTTTTTTTTTT TTTTTTTTT |
| | TTTTTTTTTTTTGCTGCCTCAGCTGCWTCTCC; |
| <SEQ ID NO: 13> | TTTTTTTTTTTTTTTTTTTTTTTTTTTTTTTTTT |
| | TTTTTTTTTTTTTTCCTTGTGCTGCCTCAGC |
| | TGC; |
| <SEQ ID NO: 14> | TTTTTTTTTTTTTTTTTTTTTTTTTTTTTTTTTT |
| | TTTTTTTTTTTTTTCACCCTCTCCTGGGCCGTGG |
| | TTTTTTT; |
| <SEQ ID NO: 15> | TTTTTTTTTTTTTTTTTTTTTTTTTTTTTTTTTT |
| | TTTTTTTTTTTTTTTTTTTTTTTTTTTTTTACCC |
| | GGACCTGTCSTTG; |
| <SEQ ID NO 16> | TTTTTTTTTTTTTTTTTTTTTTTTTTTTTTTTTT |
| | TTTTTTTTTTTTTTTTTTT TTTTTTTTTTTTTG |
| | GGCTTTCCAGAGCA; |
| <SEQ ID NO 17> | TTTTTTTTTTTTTTTTTTTTTTTTTTTTTTTTTT |
| | TTTTTTTTTTTTTTTTTTTTTTTTTTTTTTTTTT |
| | TTTTTTTCAGGCCTTCACGCTGCTG; |

or complementary or conservative nucleic acid sequences thereof, in an amount sufficient to perform a single base extension reaction on a nucleic acid sample.

40. A method of determining a CYP21 genotype, the method comprising:
(a) amplifying a DNA sample using amplification primers configured and arranged to produce distinguishable amplicons from each of a CYP21A2 gene, a CYP21A gene, a CYP21A/CYP21A2 fusion gene, and a CYP21A2/CYP21A rearranged gene; and
(b) detecting the amplicons produced in step (a), whereby said genotype is determined.

41. A method of claim 7, wherein said single nucleotide polymorphisms are identified by a method selected from the group consisting of sequencing, single strand conformational polymorphism (SSCP) detection, real-time PCR, restriction fragment length polymorphism (RFLP) detection, allele-specific oligo (ASO) hybridization, and mismatch protein binding.


SEQUENCE LISTING (26 sequences)

SEQ ID NO: 1 - DNA, 22 bp, Artificial Sequence
  Description of Artificial Sequence Primer
tccccaatccttactttttgtc

SEQ ID NO: 2 - DNA, 18 bp, Artificial Sequence
  Description of Artificial Sequence Primer
cctcaatcctctgcggca

SEQ ID NO: 3 - DNA, 21 bp, Artificial Sequence
  Description of Artificial Sequence Primer
gcttcttgatgggtgatcaat

SEQ ID NO: 4 - DNA, 18 bp, Artificial Sequence
  Description of Artificial Sequence Primer
cctcaatcctctgcagcg

SEQ ID NO: 5 - DNA, 19 bp, Artificial Sequence
  Description of Artificial Sequence Primer
ttttcccggggcaagaggc

SEQ ID NO: 6 - DNA, 24 bp, Artificial Sequence
  Description of Artificial Sequence Primer
tttccagcttgtctgcaggaggag

SEQ ID NO: 7 - DNA, 31 bp, Artificial Sequence
  Description of Artificial Sequence Primer
tttttttttttctccgaaggtgaggtaacag

SEQ ID NO: 8 - DNA, 38 bp, Artificial Sequence
  Description of Artificial Sequence Primer
tttttttttttttttttggacagctcctggaagggcac

SEQ ID NO: 9 - DNA, 46 bp, Artificial Sequence
  Description of Artificial Sequence Primer
tttttttttttttttttttttttttccccagattcagcagcgactg

SEQ ID NO: 10 - DNA, 53 bp, Artificial Sequence
  Description of Artificial Sequence Primer
ttttttttttttttttttttttttttttttttatcgccgaggtgctgcgcctg

SEQ ID NO: 11 - DNA, 61 bp, Artificial Sequence
  Description of Artificial Sequence Primer
ttttttttttttttttttttttttttttttttttttttttccatagagaagagggaycaca

SEQ ID NO: 12 - DNA, 65 bp, Artificial Sequence
  Description of Artificial Sequence Primer
tttttttttttttttttttttttttttttttttttttttttttttgctgcctcagctgcwtctcc

SEQ ID NO: 13 - DNA, 68 bp, Artificial Sequence
  Description of Artificial Sequence Primer
ttttttttttttttttttttttttttttttttttttttttttttttttccttgtgctgcctcagctgc

SEQ ID NO: 14 - DNA, 75 bp, Artificial Sequence
  Description of Artificial Sequence Primer
ttttttttttttttttttttttttttttttttttttttttttttttttcaccctctcctgggccgtggttttttt

SEQ ID NO: 15 - DNA, 81 bp, Artificial Sequence
  Description of Artificial Sequence Primer
ttttttttttttttttttttttttttttttttttttttttttttttttttttttttttttttttacccggacctgtcsttg

SEQ ID NO: 16 - DNA, 81 bp, Artificial Sequence
  Description of Artificial Sequence Primer
ttttttttttttttttttttttttttttttttttttttttttttttttttttttttttttttttttgggctttccagagca

SEQ ID NO: 17 - DNA, 93 bp, Artificial Sequence
  Description of Artificial Sequence Primer
tttttttttttttttttttttttttttttttttttttttttttttttttttttttttttttttttttttttttttcaggccttcacgctgctg

SEQ ID NO: 18 - DNA, 21 bp, Artificial Sequence
  Description of Artificial Sequence Primer
tgaccatccctctcaatcttc

SEQ ID NO: 19 - DNA, 21 bp, Artificial Sequence
  Description of Artificial Sequence Primer
tccctctttcctgccactcct

SEQ ID NO: 20 - DNA, 36 bp, Homo sapiens
ccggggtggttggcgaaggcagtcccctgtgctgcc

SEQ ID NO: 21 - DNA, 36 bp, Homo sapiens
ccggagtggttggcgaaggcagtcccctgtgctgcc

SEQ ID NO: 22 - DNA, 23 bp, Artificial Sequence
  Description of Artificial Sequence Synthetic oligonucleotide
aggtgactgccttcgccaaccac

SEQ ID NO: 23 - DNA, 23 bp, Artificial Sequence
  Description of Artificial Sequence Synthetic oligonucleotide
aggtgactgccttcgccaaccac

SEQ ID NO: 24 - DNA, 24 bp, Artificial Sequence
  Description of Artificial Sequence Synthetic oligonucleotide
aggtgactgccttcgccaaccact

SEQ ID NO: 25 - DNA, 23 bp, Artificial Sequence
  Description of Artificial Sequence Synthetic oligonucleotide
aggtgactgccttcgccaaccac

SEQ ID NO: 26 - DNA, 23 bp, Artificial Sequence
  Description of Artificial Sequence Synthetic oligonucleotide
aggtgactgccttcgccaaccac